(19) United States
(12) Patent Application Publication
(10) Pub. No.: US 2023/0123148 A1
(43) Pub. Date: Apr. 20, 2023

(54) TARGETING QUORUM-SENSING PEPTIDES FOR DIAGNOSIS, TREATMENT AND/OR PREVENTION OF COLORECTAL CANCER METASTASIS

(71) Applicant: UNIVERSITEIT GENT, Gent (BE)

(72) Inventors: Bart DE SPIEGELEER, Gent (BE); Evelien WYNENDAELE, Herzele (BE); Christophe VAN DE WIELE, Gent (BE); Olivier DE WEVER, Destelbergen (BE); Nathan DEBUNNE, Moerzeke (BE)

(21) Appl. No.: 17/907,348

(22) PCT Filed: Mar. 26, 2021
(86) PCT No.: PCT/EP2021/057976

§ 371 Date: Sep. 26, 2022

(30) Foreign Application Priority Data
Mar. 27, 2020 (EP) 20166415.8
Jan. 6, 2021 (EP) 21150360.2

(51) Int. Cl. A61K 35/747; G01N 33/574; A61P 35/04; G01N 33/68

(52) U.S. Cl. CPC A61K 35/747; G01N 33/57419; A61P 35/04; G01N 33/6848; G01N 2333/335

(57) ABSTRACT

Quorum-sensing peptides are provided as diagnostic biomarkers, and quorum-sensing peptide inhibiting substances are provided for use in the treatment of metastasis of colorectal cancer in a subject, in particular a human subject. Methods for reducing metastasis of colorectal cancer in a subject include administering to the subject a microorganism that reduces or blocks activity of a pro-metastatic quorum-sensing peptide or a metabolite thereof present in a gastrointestinal tract or blood of the subject; and/or that reduces or blocks production of the pro-metastatic quorum-sensing peptide or the metabolite thereof in the gastrointestinal tract or blood of the subject. The pro-metastatic quorum-sensing peptide may include EntF or the metabolite EntF* of EntF.

### CROSS-REFERENCES TO RELATED APPLICATIONS

This application is a national-stage application under 35 U.S.C. § 371 of International Application No. PCT/EP2021/057976, filed Mar. 26, 2021, which International Application claims benefit of priority to European Application No. 20166415.8, filed Mar. 27, 2020 and European Application No. 21150360.2, filed Jan. 6, 2021.

### SEQUENCE LISTING

A computer-readable form (CRF) sequence listing having file name GHE0049PA-SEQlisting.txt (3548 bytes), created Sep. 26, 2022, is incorporated herein by reference. The nucleic acid sequences and amino acid sequences listed in the accompanying sequence listing are shown using standard abbreviations as defined in 37 C.F.R. § 1.822.

### TECHNICAL FIELD

The present disclosure relates to quorum-sensing peptides as diagnostic biomarkers, and to quorum-sensing peptide inhibiting substances for use in the treatment and/or prevention of metastasis of colorectal cancer in a subject; in particular a human subject.

### BACKGROUND

Growing evidence obtained in the last decade suggests that the gut microbiota influences the health of the host. For example, by comparing faeces from healthy persons and patients, higher abundances of Enterococcus, Escherichia and Fusobacterium species were observed in multiple intestinal disorders, including colorectal cancer (CRC) and Crohn's disease. However, the causative factors for diseases development or progression are not well understood and current research is mainly limited to bacterial-derived short-chain fatty acids and amino-acid derived amines.

Quorum sensing peptides are traditionally regarded as intra- and inter-bacterial communication molecules, but given their wide structural variety and co-evolution, we anticipate that these molecules may also interact with the host. Up till now, however, quorum sensing peptides have not yet been unambiguously demonstrated to be present in biofluids. Only an indirect indication of the in vivo presence of an unidentified quorum sensing peptide was described in the stool of patients suffering from Clostridium difficile infection. Previously, the researchers of the present disclosure focused on Enterococcus faecium, one of the most abundant species in the human intestinal microbiota, which synthesizes the enterocin induction factor, i.e. the propeptide of the EntF quorum sensing peptide (AGTKPQGKPASNLVECVFSLFKKCN; SEQ ID NO:2). This peptide serves as a communication signal, regulating the production of enterocin A and B toxins, which are produced to inhibit the growth of similar or closely related bacterial strains. Standard protein BLAST searches thereby indicate that EntF-producing E. faecium strains indeed are present in different human faeces samples (Table 1). On the other hand, our experimental data indicated that not all E. faecium strains have this EntF gene in their genome (Table 2), indicative for the inter-individual variation in the (gut) microbial community.

Previously, it was demonstrated that metabolization of EntF in faeces and colonic tissue homogenate yields a 15-mer peptide EntF* (SNLVECVFSLFKKCN; SEQ ID NO:1). This quorum sensing peptide was found to promote angiogenesis and tumor cell invasion in in vitro experiments using HCT-8 CRC cells (Wynendaele et al., Peptides 2015, 64: 40-48).

In the present disclosure, the inventors show that the EntF* quorum sensing peptide can be detected and targeted in the gastro-intestinal tract and/or the circulation of a subject. More specifically, the inventors demonstrate for the first time that quorum sensing peptides, and in particular the quorum-sensing peptide EntF*, promotes in vivo colorectal cancer metastasis, and has pro-metastatic properties in vivo.

### SUMMARY

Based on the finding that quorum-sensing peptides, and in particular the pro-metastatic EntF* quorum-sensing peptide, promote cancer-metastasis, the present application relates to the use of quorum-sensing targeting substances for the treatment and/or prevention of metastasis of colorectal cancer. Said quorum-sensing targeting substances can be peptide inhibitors, peptide antagonists, molecules that degrade quorum-sensing peptides, or pre-, pro- or synbiotics, such as e.g. (engineered) micro-organisms, that are unable to synthesize the metastasis-promoting quorum sensing peptides and that reduce or inhibit the functionality of the targeted quorum-sensing peptide or the production of the quorum-sensing peptide by gut microbiota.

The present disclosure is thus directed to a quorum-sensing inhibiting substance, in particular a microorganism, for use in the treatment, reduction and/or prevention of metastasis of colorectal cancer in a subject. Typical for various embodiments is that said quorum-sensing inhibiting substance reduces and/or inhibits the activity and/or production of one or more pro-metastatic quorum-sensing peptides and/or their metabolites in the gastro-intestinal tract and/or blood of said subject. In a particular aspect, the pro-metastatic quorum-sensing peptide is derived from enterocin induction factor (SEQ ID NO:3, MEEKNRLNAKQCSDQELKKIKGGAGTKPQGKPASNLVECVFSLFKKCN). More specifically, the pro-metastatic quorum-sensing peptide is the peptide EntF (SEQ ID NO:2, AGTKPQGKPASNLVECVFSLFKKCN) and/or the EntF metabolite EntF* (SEQ ID NO:1, SNLVECVFSLFKKCN). Since it was shown for the first time in the present disclosure that quorum-sensing peptides are able to promote in vivo colorectal cancer metastasis, inhibition or degradation of said quorum-sensing peptides is a novel treatment strategy to prevent and/or reduce the presence and/or development of colorectal cancer metastasis.

In one aspect, the present disclosure is thus directed to a quorum-sensing inhibiting substance for use in the treatment and/or prevention of metastasis of colorectal cancer, wherein said quorum-sensing inhibiting substance reduces and/or inhibits the activity of a quorum-sensing peptide and/or its metabolites in the gastro-intestinal tract and/or blood of the subject. In one embodiment, the quorum-sensing inhibiting substance is a microorganism as provided herein.

In a further embodiment, the quorum-sensing inhibiting substance according to the present disclosure is a product or compound that reduces, inhibits or blocks the production and/or activity of the one or more quorum-sensing (pro)peptide(s) in the gastro-intestinal tract and/or the blood or serum of the subject. In an even more preferred embodiment, said product is a compound or peptide having antagonistic activity to the pro-metastatic quorum-sensing peptide. In still a further embodiment, the quorum-sensing inhibitor of the present disclosure targets the peptide EntF (SEQ ID NO:2) and/or the peptide EntF* (SEQ ID NO:1), in particular EntF*. In an even further embodiment, the quorum-sensing inhibiting substance targets the first, second and/or tenth amino acid of the quorum-sensing peptide EntF* (SEQ ID NO:1).

In another aspect, the present disclosure is directed to a quorum-sensing inhibiting substance for use in the treatment and/or prevention of metastasis of colorectal cancer, wherein said quorum-sensing inhibiting substance is a quorum-quenching agent that degrades a quorum-sensing peptide; in particular that degrades the quorum-sensing peptide EntF*. In a further embodiment, said quorum-sensing inhibiting substance is an enzyme that degrades the quorum-sensing peptide, such as pepsin, trypsin, chymotrypsin, papain, bromelain, serrapeptase, pancrelipase. In the alternative, said quorum-sensing inhibiting substance is an enzyme activator that selectively activates endogenous proteases and/or peptidases. These agents can be used as such or as a targeted-delivery formulation.

In still another aspect, the present disclosure is directed to a quorum-sensing inhibiting substance for use in the treatment and/or prevention of metastasis of colorectal cancer in a subject, wherein the quorum-sensing inhibiting substance is a microorganism, in particular a bacterial strain that does not produce the enterocin induction factor, and in particular the quorum-sensing peptide EntF (SEQ ID No: 1) and/or its metabolite EntF* (SEQ ID No: 2), e.g. as determined by qPCR and/or UHPLC-MS/MS and/or any other established technique. More specific, the bacterial strain is lacking the EntF gene or is genetically modified, e.g. by mutating or deletion of part or all of the EntF gene. In a further embodiment, said bacterial strain is selected from the order lactic acid bacteria (Lactobacillales), and more specific from the genus Enterococcus; preferably said bacterial strain is E. faecium LMG15710, NCIMB10415(SF68), W54, LMG S-28935, THT020101, or any other strain where absence of EntF and/or EntF* has been proven by qPCR and/or UHPLC-MS/MS and/or any other established technique. In still a further embodiment, the bacterial strain that does not produce the quorum-sensing peptide EntF and its metabolite EntF*, is administered to the subject and reduces the production of the quorum-sensing peptide EntF by other bacterial strains in the gastro-intestinal tract e.g. by altering the bacterial flora and/or suppressing the levels of the EntF/EntF* producing strains. In other words, in one aspect of the present disclosure, administration of the quorum-sensing inhibiting substance reduces the production of the quorum-sensing peptide EntF by other bacterial strains in the gastro-intestinal tract.

In a further aspect, the presence of bacterial strains producing the quorum-sensing peptide EntF is reduced or prevented when the quorum-sensing inhibiting substance, in particular the bacterial strain that does not produce the quorum-sensing peptide EntF, is administered to the subject, e.g. as (part of) a probiotic or medicine.

In the different embodiments of the present disclosure, a quorum-sensing targeting or inhibiting substance, such as a microorganism, for use in the treatment and/or prevention of metastasis of colorectal cancer is administered to a subject. In a further embodiment, said subject is a mammal. In an even further embodiment, the subject is a human subject. In yet another embodiment, the subject is a human subject diagnosed with a colorectal primary tumour. In still another embodiment, the subject is a human subject diagnosed with or at risk of colorectal cancer metastasis.

In another aspect, the present disclosure discloses a method of treating, reducing and/or preventing metastasis of colorectal cancer in a subject, wherein a therapeutically effective amount of a quorum-sensing inhibiting substance is administered to the subject and wherein said quorum-sensing inhibiting substance reduces and/or inhibits the activity and/or production of a pro-metastatic quorum-sensing peptide and/or its metabolites in the gastro-intestinal tract and/or blood of said subject. The quorum-sensing inhibiting substance in said method is a quorum-sensing inhibiting substance according to all the different embodiments as described herein. In a particular embodiment, the quorum-sensing inhibiting substance is a microorganism, such as e.g. a bacterial strain as provided herein.

The methods according to all the different embodiments of the present disclosure may further comprise a step of adjusting the diet of the subject to prevent and/or reduce the uptake and/or presence of Enterococcus faecium producing a quorum-sensing peptide; in particular a quorum-sensing peptide that promotes colorectal cancer metastasis. In a further embodiment, the methods may comprise a step of adjusting the diet of the subject to prevent and/or reduce the uptake and/or presence of Enterococcus faecium producing the EntF quorum-sensing peptide.

In still another aspect of the present disclosure, a method of preventing, treating and/or reducing metastasis of colorectal cancer in a subject is disclosed, wherein the method comprises the following step: detecting the level of a quorum-sensing peptide in a sample derived from a subject, and based on the outcome, adjusting the diet of said subject to prevent and/or reduce the uptake and/or presence of Enterococcus faecium producing the quorum-sensing peptide.

In a further embodiment, a method for preventing, treating and/or reducing metastasis of colorectal cancer in a subject is disclosed wherein the method comprises the steps of detecting the level of the quorum-sensing peptide EntF (SEQ ID NO:2) in a sample derived from the subject, and based on the outcome adjusting the diet of said subject to prevent and/or reduce the uptake and/or presence of Enterococcus faecium producing the quorum-sensing peptide EntF. In another aspect, a method for preventing, treating and/or reducing metastasis of colorectal cancer in a subject is disclosed wherein the method comprises the steps of detecting the level of the EntF metabolite EntF* (SEQ ID NO:1) in a sample derived from the subject, and based on the outcome adjusting the diet of said subject to prevent and/or reduce the uptake and/or presence of Enterococcus faecium producing the quorum-sensing peptide EntF.

In all said methods of the present disclosure, the level of a quorum-sensing peptide is detected in (a sample of) a subject; preferably a mammal; even more preferably a human subject.

In a further aspect, the present disclosure is also directed to a composition, including its use as a medicament, comprising an EntF*-antagonistic peptide, a molecule derived from an EntF*-antagonistic peptide, an Enterococcus strain not producing EntF/EntF*, or a genetically modified Enterococcus strain wherein part or all of the gene producing EntF/EntF* has been deleted or mutated, and a pharmaceutically acceptable carrier. In one embodiment, the Enterococcus strain is an Enterococcus faecium strain.

In another embodiment, the present disclosure is directed to a diagnostic method for analyzing the presence of quorum-sensing peptides in a sample of a subject to assess the presence of such peptides that influence tumor metastasis in a subject having colorectal cancer.

Screening can be done by analyzing feces and/or blood or serum, other body-fluids, and/or other samples.

### BRIEF DESCRIPTION OF THE DRAWINGS

With specific reference now to the figures, it is stressed that the particulars shown are by way of example and for purposes of illustrative discussion of the different embodiments of the present disclosure only. They are presented in the cause of providing what is believed to be the most useful and readily description of the principles and conceptual aspects of the present disclosure. In this regard no attempt is made to show structural details of the present disclosure in more detail than is necessary for a fundamental understanding of the present disclosure. The description taken with the drawings making apparent to those skilled in the art how the several forms of the present disclosure may be embodied in practice.

FIGS. 1A-1I. In vitro formation and in vivo presence of the EntF quorum sensing peptide derived metabolite.

FIG. 1A Sequence of the quorum sensing propeptide enterocin induction factor, the mature quorum sensing peptide EntF and its metabolite EntF*.

FIG. 1B The in vitro formation rate of EntF* from EntF in colon and faeces homogenate. Bars represent mean formation rate ±s.e.m from 6 (colon), resp. 4 (faeces) independent experiments. Statistically significant differences were determined by a Mann-Whitney U test with indicated p-values.

FIG. 1C Effect of alanine-derived EntF* analogues on E-cadherin expression. Ranking in five classes (lighter grey/lower part to darker grey/upper part: increasing significance) was performed using the Fisher's LSD p-values, which was confirmed using the Jenks natural break algorithm. Based on ranking, it was proven that the first, second and tenth amino acid are the most important amino acids for the epithelial-mesenchymal promoting (EMT) effects of EntF*.

FIG. 1D CaCo-2 apparent permeability coefficients (Pₐₚₚ) of 3 different quorum sensing peptides. Bars represent mean Pₐₚₚ-values ±s.e.m. (n=5-6 independent experiments per group); the shaded area represents the limit of detection.

FIG. 1E Flow chart of data acquisition, from sampling of serum samples to detection and confirmation of EntF*. Different LC-MS methods: reversed-phase ultra-high-performance liquid chromatography (RP-UPLC) using triple quadrupole (TQ) in MRM mode (LC₁-MS₁), high-resolution quadrupole time-of-flight (LC₁-MS₂), high-resolution quadrupole-orbitrap (LC₁-MS₃) and HILIC-amide UPLC using TQ in MRM mode (LC₂-MS₁). qPCR was performed on faeces samples from those mice to demonstrate the presence of the EntF* containing E. faecium.

FIG. 1F Chromatographic profile of (1) negative serum sample; (2) positive serum sample; (3) serum of mice i.p. injected with EntF*; all using RP-UPLC with detection by electrospray ionization mass spectrometry (ESI-MS) using TQ in MRM mode (m/z=865→202.08+315.17).

FIG. 1G Chromatographic profile of (1) negative serum sample; (2) positive serum sample; (3) serum of mice i.p. injected with EntF*; all using HILIC amide UPLC with detection by ESI-MS using TQ in MRM mode (m/z=865→202.08+315.17).

FIG. 1H Isotopic distribution of the double charged EntF* measured in a positive serum sample using RP-UPLC with detection by ESI-MS using quadrupole-orbitrap.

FIG. 1I High-resolution tandem mass spectrum of EntF* with characteristic fragments, using RP-UPLC with detection by Q-TOF.

FIG. 2. Effect of EntF* on E-cadherin expression. A significant mean decrease of 36% in E-cadherin level for EntF* in comparison with placebo was observed (one-way ANOVA, Fisher's LSD).

FIG. 3. Verification of the applied LC₁-MS₁ method. The calibration curve was constructed out of 7 independent measurements with independent sample preparation of a serum sample spiked at 3 different concentrations: 100 pM (n=4), 250 pM (n=1) and 1 nM (n=2). The best-fitted regression line represents the calibration curve with indicated R²-value. The accuracy (±17.1%) and the precision (RSD=+10.3%) were determined out of the QC samples measured at 500 pM (n=3). A precision of 31.7% was measured at the limit of quantification. The shaded bar represents the area measured in negative samples.

FIGS. 4A-4G. In vivo metastasis-inducing effect of EntF* in an orthotopic colorectal cancer mouse model.

FIG. 4A Experimental schematic timeline. Female Swiss nu/nu mice were orthotopically injected with 1×10⁶ luciferase transfected HCT-8 cells at the age of 5 weeks. During 6 weeks, the mice were daily i.p. injected with 100 nmol kg⁻¹ EntF*, PBS control or 0.1 mg kg⁻¹ EGF control. Bioluminescent imaging was performed weekly to determine cancer progression. After 6 weeks, the mice were euthanized and the caecum, liver and lungs collected.

FIG. 4B A representative image comparing the basal bioluminescence activity between the three treatments. Mice were i.p. injected with 150 mg kg⁻¹ luciferine and imaged 10 minutes later in the supine position.

FIG. 4C Tumour growth curves of the three groups. Based on linear regression slope comparison, the EntF* as well as the positive control EGF treatment resulted in a significant increase of tumour growth compared to the control with indicated p-values. Data represent mean fold change ±s.e.m. (n=17-18 mouse per group).

FIG. 4D Macroscopic, representative caecum pictures of the three treatments at the end of the experiment.

FIG. 4E Caecum tumour nodules were counted and the data represent the mean±s.e.m. Statistically significant differences were determined by a Mann-Whitney U test (n=15-38 per group) with indicated p-values.

FIG. 4F Histopathological scores on haematoxylin and eosin (H&E) stainings of the liver with statistically significant differences determined by a Mann-Whitney U test (n=8 for PBS, n=30 for EntF*, n=9 for EGF) with indicated p-values.

FIG. 4G Histopathological scores on H&E stainings of the lungs with statistically significant differences determined by a Mann-Whitney U test (n=8 for PBS, n=30 for EntF*, n=9 for EGF) with indicated p-values.

FIG. 5. Daily exposure comparison between EntF*-treated Swiss nu/nu mice and non-peptide treated Swiss nu/nu mice. The daily exposure in the female Swiss nu/nu mice (n=65 placebo mice, with negative mice (<LOQ=100 pM) set as 0 pM) (black) and the female Swiss nu/nu mice after injection with EntF* (gray, n=14) is given. Error bars represent s.e.m. values. Exposure was calculated from pharmacokinetic data after i.p. injection of EntF* for 0-180 min (EntF* treatment) or from steady-state endogenous presence of the peptide. The daily injections of 100 nmol kg⁻¹ in the bioluminescent metastasis experiment gave daily exposures which were five times higher than the endogenous (natural) exposure in those mice.

FIGS. 6A and 6B The antagonistic effects of Nef-M1 and QSP76S1A on the E-cadherin reducing effect of QSP76 on HCT-8 cells. Statistically significant differences were determined by a one-sided student's t test. FIG. 6A. Nef-M1 (an antagonist to the endogenous ligand SDF-1 or CXCL12 of the CXC chemokine receptor 4 (CXCR4)) abolishes and FIG. 6B. QSP76S1A (where Serine at position 1 is replaced by alanine) reduces the in vitro pro-metastatic effect of QSP76 (EntF*).

FIG. 7 Gene information of EntF and EntF* and used primers for qPCR. The peptide sequence (MEEKNRLNAKQCSDQELKKIKGGAGTKPQGKPA SNLVECVFSLFKKCN; SEQ ID NO:3) was retrieved using the Uniprot database (several strains such as for example R2NMY7_ENTFC, A0A0M2B1G5_ENTFC, A0A0M1XX59_ENTFC, S4DWX5_ENTFC, J6Y1Q0_ENTFC, U2QBF0_ENTFC, J6Y2H7_ENTFC, and Q9X472_ENTFC, A0A4Y3JRR7_9ENTE) Nucleic Acids Res. 47: D506-515 (2019)).

FIG. 8: Effects of EntF* (1 μM) and CXCL12 (10 ng/mL) on relative E-cadherin expression (versus placebo) in different CRC cell lines (Mean±SEM, n=6-15).

FIG. 9: The experimental overview to evaluate the potential of non-EntF producing strains (treatment) to reduce the harmful EntF producing strains in mice.

FIG. 10: qPCR determined EntF*-copies per gram faeces determined over time (before treatment, after 1 day of treatment, after 1 week of treatment and 1 week post treatment) in the placebo and test group. Statistically significant differences were determined using a post-hoc Bonferroni's multiple comparisons test.

FIG. 11: Colon content concentrations of EntF found in the placebo and LBP treated groups. The error bars around the average represent the SEM and the shaded area represents the limit of detection. Statistical significance (n=4) is determined using a student t test.

FIG. 12: Serum concentrations of EntF* found in the placebo and test LBP groups. The error bars around the average represent the SEM and the shaded area represents the limit of detection. Statistical significance (n=4) is determined using a student t test.

### DETAILED DESCRIPTION

In the present disclosure, the inventors show that the EntF* quorum sensing peptide can be detected and targeted in the gastro-intestinal tract and/or the circulation of a subject. More specifically, the inventors demonstrate for the first time that quorum sensing peptides, and in particular the quorum-sensing peptide EntF*, promote in vivo colorectal cancer metastasis, and has pro-metastatic properties in vivo.

In a first aspect, a quorum-sensing inhibiting substance for use in the treatment and/or prevention of metastasis of colorectal cancer is thus provided. Said quorum-sensing inhibiting substance reduces and/or inhibits the activity and/or production of enterocin induction factor in bacteria, and more specific of one or more pro-metastatic quorum sensing peptides or metabolites thereof. As used herein, “pro-metastatic” means that said peptides or metabolites promote or induce metastasis of cancer cells from the primary tumor in vivo. A representative in vivo model to determine pro-metastatic activity is provided herein. Said pro-metastatic peptides are for example the EntF peptide (SEQ ID No: 2) and the metabolite thereof, i.e. EntF* peptide (SEQ ID No: 1). The “inhibiting substances” or “inhibitor” can be found by commonly applied screening techniques known to the skilled person, and include:
peptide antagonists (peptides with an antagonistic activity),
molecules that degrade quorum-sensing (pro)peptides such as e.g. enzymes,
engineered microorganisms that are unable to synthesize the pro-metastatic quorum sensing (pro)peptides and/or that reduce or inhibit the functionality of said quorum-sensing (pro)peptides, and
bacterial strains that are unable to synthesize the pro-metastatic quorum sensing (pro)peptides and in particular that are able to reduce or inhibit the production of the pro-metastatic quorum-sensing peptides by gut microbiota.

In one embodiment, the quorum-sensing inhibiting substance is thus an antagonistic compound to the pro-metastatic peptide or metabolites thereof. As used herein, an “antagonistic compound” “antagonist” refers to any chemical compound or biological molecule has antagonistic activity and/or that impairs, in particular decreases, the ability of such targeted quorum-sensing peptide as defined herein, to respond or bind to their respective ligand or receptor such as e.g. the CX chemokine receptor 4 (CXCR4); or to impair its functionality such as the epithelial-mesenchymal promoting (EMT) activity or angiogenesis.

In one aspect, the quorum-sensing inhibiting substance is a product or compound that specifically binds the peptide EntF* and decreases its activity or functionality. For example, the quorum-sensing inhibiting substance is a compound that targets the EntF* peptide, for example a compound that targets or specifically binds the first, second and/or tenth amino acid of the quorum-sensing peptide EntF*.

In one embodiment, said inhibitor includes antibodies and antigen-binding fragments thereof. In the alternative, EntF or EntF* binding moieties or antagonists can be used which include a variety of different types of molecules including those that specifically bind resp. EntF or EntF*, in particular the first, second and tenth amino acid of the peptide EntF* which have been shown to be involved in the EMT activity of the peptide. Such ligands include small molecules, polypeptides (e.g. a fusion protein), antibodies, or nucleic acids, and the like. The term “antibody” refers to polyclonal antibodies, monoclonal antibodies (mAbs), chimeric antibodies, humanized antibodies, a human engineered antibody, a human antibody, as well as antigen binding antibody fragments, a domain antibody (dAb), heavy chain antibodies (hcAb), minibodies, a variable domain of camelid heavy chain antibody (VHH or Nanobody®), a variable domain of the new antigen receptor (VNAR) and engineered CH2 domains (nanoantibodies), and molecules having antigen binding functionality. EntF* antibodies and methods of making the same are well known in the art. As demonstrated herein, the peptide Nef-M1 blocks the effect of EntF* on the CXCR4 receptor and is thus an example of a quorum-sensing inhibiting substance.

The effect on EMT activity of EntF* (also referred herein as QSP76) was modulated by alanine-scans. The results show the importance of certain amino acid residues for the biological activity on EMT, in particular residues on position one, two and ten of EntF*. Changing amino acids is a flexible way of increasing as well as of decreasing the activity, and said method can be used to design antagonistic peptides. In one embodiment, the antagonistic peptide is characterized by the following sequence: X₁X₂LVECVFSX₃FKKCN (SEQ ID NO:4), wherein is X₁ is an amino acid other than S, and/or wherein X₂ is an amino acid other than N and/or X₃ is an amino acid other than L.

Hence in one embodiment, the antagonist is an oligopeptide analogous to the quorum sensing peptide EntF* but different at positions one, two and/or ten, in particular at position one. As demonstrated herein, the EntF* homologues peptide QSP76S1A (ANLVECVFSLFKKCN, SEQ ID NO:5) goes in competition with EntF* on the CXCR4 receptor and is thus an example of a EntF* antagonist.

In a further embodiment, the quorum-sensing inhibiting substance is a quorum-quenching agent that degrades a quorum-sensing (pro)peptide, and as such inhibits or prevents its activity. For example this quorum-quenching agent is an enzyme, for example pepsin, trypsin, chymotrypsin, papain, bromelain, serrapeptase, pancrelipase. On the other hand, the quorum-sensing inhibiting substances can also be an enzyme activator that selectively activates endogenous proteases and/or peptidases.

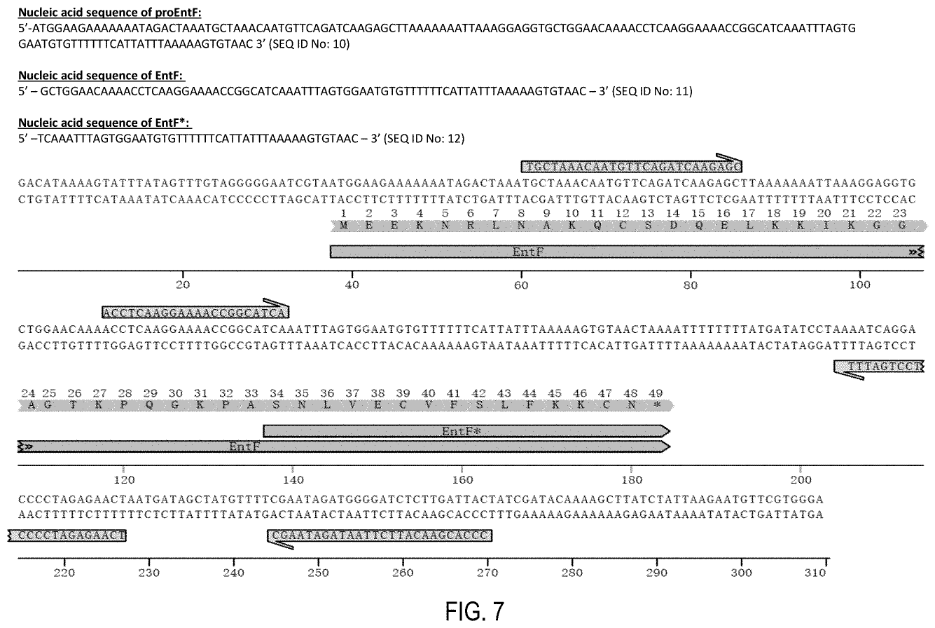

In another aspect, the quorum-sensing inhibiting substance can also be a microorganism, in particular a bacterial strain that does not produce the quorum-sensing peptide EntF*. Said bacterial strain can for example be genetically modified to lack the EntF family enterocin induction factor gene as e.g. defined by accession no NC_021994.1 or as provided in FIG. 7 (SEQ ID Nos 10, 11 and 12), or a gene having at least 80% identity thereto, in particular at least 85%, 90%, 910%, 92%, 93%, 94%, 95%, 96%, 97%, 98% or 99% identity thereto (e.g. NC_020207.1 or NC_017960.1). Typically, the genetic modification results in a non-functional EntF gene, e.g. by mutation or deletion in the EntF gene, in particular a deletion of the full EntF gene. As a further option, the bacterial strain is a wild-type strain that does not produce a functional EntF or EntF* peptide and/or is lacking the EntF gene. In one embodiment, the bacterial strain is from the order Lactobacillales, more specific from the genus Enterococcus or Lactobacillus. In a further embodiment, the bacterial strain is an Enterococcus faecium strain.

A bacterial strain not producing a functional EntF or EntF* peptide and/or lacking the EntF gene can for example be the E. faecium LMG15710 (ATCC 6569), NCIMB10415 (E. faecium SF68; DSM 10663 commercially available—EU authorized feed additive for animals), W54 (Strasser et al., Nutrients 2016; 8(11)), LMG S-28935 (Chisari et al. Curr Clin Pharmacol 2017; 12(2): 99-105), THT020101, or any other (Enterococcus) strain where absence of EntF and/or EntF* has been proven by qPCR and/or UHPLC-MS/MS and/or any other established technique, and in particular by Liquid chromatography-mass spectrometry (LC-MS) under the same conditions as demonstrated herein.

As shown for the first time in the present disclosure, said bacterial strains are able to alter the bacterial flora of the subject and/or to overgrow or to suppress the levels of the EntF* producing strains in the subject.

The bacterial strains can be administered to the subject orally, in all possible forms such as formulated in a powder, tablet, capsule, or any other way known to the skilled person. In one embodiment, the bacterial strain is administered as part of a probiotic composition, for example via the daily food or via a specific pharmaceutical composition.

In a further aspect, the diet of the subject can be adjusted so that it comprises a quorum-sensing inhibiting substance that prevents and/or reduces the uptake and/or presence of quorum-sensing (pro)peptides in the subject. For example, the substance can prevent and/or reduce the uptake and/or presence of Enterococcus faecium producing the quorum-sensing (pro)peptides in the subject.

In the methods provided herein, the quorum sensing inhibiting substance is used or administered to the subject in an effective amount. The terms “effective amount” or “effective dose” are used interchangeably herein and denote an amount of the pharmaceutical active inhibiting substance of the present disclosure which has a prophylactically or therapeutically relevant action on cancer metastasis, in particular on colorectal cancer metastasis. The spread of cancer from its tissue of origin and its subsequent growth in other organs is the most life-threatening aspect of the disease. This process is called metastasis, and requires cancer cells to survive and proliferate outside their tissue of origin. The first crucial step in this process is the invasion of cancer cells into tissue surrounding the tumour and the vasculature.

The quorum sensing inhibiting substance of the present disclosure is capable to prevent and/or reduce cancer cell dissemination and metastasis formation. As such, the present disclosure relates to the quorum sensing inhibiting substance described herein for use in treating cancer by inhibiting the invasion of tumour cells into surrounding or adjacent tissue, cells and/or their entry into the circulatory system. Hence the substances or compounds of the present disclosure are especially useful for inhibiting or stopping tumour spread or cancer metastasis, in particular colorectal cancer i.e. the development of cancer from the colon or rectum (parts of the large intestine, including rectal cancer, colon cancer and bowel cancer). In a specific embodiment, the quorum sensing inhibiting substance prevents or reduces colon cancer metastasis. In a further embodiment, the tumour or cancer is a CXCR4 positive tumour or cancer, i.e. whereby the cancer cells express CXCR4 as e.g. can be determined using different techniques known to the skilled person, such as flow cytometry, qPCR analysis and/or western blotting. C-X-C chemokine receptor type 4 (CXCR-4) also known as fusin or CD184 (cluster of differentiation 184) is a protein that in humans is encoded by the CXCR4 gene. While CXCR4's expression is low or absent in many healthy tissues, it was demonstrated to be expressed in over 23 types of cancer, including breast cancer, ovarian cancer, melanoma, and prostate cancer. Expression of this receptor in cancer cells has been linked to metastasis to tissues containing a high concentration of CXCL12, such as lungs, liver and bone marrow.

Generally, for pharmaceutical use, the quorum sensing inhibiting substance of the present disclosure may be formulated as a pharmaceutical preparation or pharmaceutical composition comprising at least one quorum sensing inhibiting substance of the present disclosure and at least one pharmaceutically acceptable carrier, diluent or excipient and/or adjuvant, and optionally one or more further pharmaceutically active compounds. Such a formulation may be in a form suitable for oral administration, for administration by suppository, for parenteral administration (such as by intravenous, intramuscular or subcutaneous injection or intravenous infusion), for intranasal, transdermal, transmucosal, rectal or pulmonary administration.

Examples of such formulations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration. Carriers, excipients, and diluents that are suitable for such formulations are e.g. lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof.

This present disclosure further provides a pharmaceutical composition comprising a quorum sensing inhibiting substance according to this present disclosure and a pharmaceutically acceptable carrier, diluent and/or excipient, and the uses thereof as provided herein.

The present disclosure also provides an in vitro method for the detection of a quorum-sensing peptide or metabolite thereof in a sample from a subject, in particular a subject diagnosed with cancer, more in particular diagnosed with colorectal cancer. In one embodiment, this method comprises (1) preparing the sample by using C18 or hydrophilic interaction liquid chromatography (HILIC) solid-phase extraction, preferably HILIC-amide solid-phase extraction, (2) performing liquid chromatography on the sample prepared in step (1) wherein the gradient conditions are such that loss of the analyte, e.g. by adsorption, is minimized, such as for example by the choice of a suitable start mobile phase and/or gradient slope, and (3) detection of the peptide or metabolite using a suitable detection method, such as a MS-based method. In one embodiment, the mobile phase consists of water:acetonitrile:DMSO (V/V) containing 0.05%-1% of an acid, in particular formic acid. The flow rate is set to 0.1-1 mL/min, in particular 0.5 mL/min. In a further embodiment the gradient program starts with 70%-95% of the mobile phase, in particular 75-85%, for about 0.5 to 1.5 min, followed by a linear gradient to 30-50%, in particular 35-45% of the mobile phase for 2.5-4 minutes, in particular 3-4 minutes. After that the gradient is decreased to 10-20% mobile phase, in particular about 15%, for 4-6 min.

In another aspect of the present disclosure, the detection of a quorum-sensing peptide or metabolite thereof, in particular EntF and/or EntF*, can be used in the diagnosis of colorectal metastasis in a subject.

More specific, the present disclosure is directed to a diagnostic method for analyzing the presence of quorum-sensing peptides in a sample of subject to assess the presence of such peptides that influence tumor metastasis in a subject having colorectal cancer.

Screening can be done by analyzing feces and/or blood, other body-fluids, and/or other samples.

The present disclosure furthermore relates to the use of the knowledge obtained by the diagnostic method in order to aid in the differential patient classification and in prognosis, as well as to influence the progression of cancer metastasis, by for example providing beneficial bacteria, providing antagonists for harmful quorum sensing peptides and other measures as provided herein.

The present disclosure furthermore relates to a method of detecting a patient at risk of colorectal cancer metastasis, said method comprising detecting the presence and/or amount of the quorum-sensing peptide EntF (e.g. in faeces) and/or EntF* (e.g. in plasma) in a sample of said subject.

In a further aspect, the current disclosure relates to the use of the knowledge obtained by the diagnostic method in order to reduce or prevent cancer metastasis in a patient diagnosed with colorectal cancer, by for example providing bacteria that produce non-harmful quorum sensing peptides and/or providing antagonists for harmful quorum-sensing peptides.

The present disclosure in particular relates to microorganisms, having neutral quorum sensing peptides to replace in the gut the strains that have quorum sensing peptides shown herein to negatively influence metastasis, such as the EntF* peptides. In one embodiment, these microorganisms are probiotica (bacterial strains that are considered healthy for the gut).

More specific, the present disclosure provides a method of preventing, risk-evaluation, diagnosis, treating and/or reducing metastasis of colorectal cancer in a subject, said method comprising the steps of:
detecting the presence and level of one or more selected pro-metastatic quorum-sensing peptides or their metabolites; in particular the pro-metastatic quorum-sensing peptide EntF (SEQ ID NO:2) and/or EntF* (SEQ ID NO:1), in a sample derived from said subject;
based on the outcome, which can also serve as a diagnostic biomarker, administering one or more of the quorum sensing inhibiting substance as defined herein before or adjusting the diet and/or lifestyle of said subject, to prevent and/or reduce the uptake and/or presence of Enterococcus faecium producing said quorum-sensing peptide.

### Examples

### Materials and Methods

Homogenate preparation. Krebs-Henseleit (KH) buffer (pH 7.4) (Sigma-Aldrich, Belgium) was prepared by dissolving the powdered medium in 900 mL water while stirring. To this solution, 0.3790 g CaCl₂×2H₂O and 2.098 g NaHCO₃ are subsequently added while stirring. NaOH or HCl was used to adjust to pH 7.4. This solution was then further diluted to 1000 mL using ultrapure water.

For the preparation of colon tissue homogenate, two colons were collected from two C57BL/6 female mice after cervical dislocation. After cleaning and rinsing the organs using ice-cold KH buffer, the colons were cut in little pieces and transferred into a 15 mL tube to which 5 mL ice-cold KH buffer was added. The colons were then homogenized for 1 minute. After the larger particles were allowed to settle for about 30 minutes at 5° C., approximately 2 mL of the middle layer was dispensed into a 2 mL Eppendorf tube, and stored at −35° C. until use. Just before use, the homogenate was diluted to a protein concentration of 0.6 mg/mL.

Faeces homogenate was prepared by collecting faeces from two C57BL/6 female mice after cervical dislocation. The same procedures as with the colon tissue were performed. Just before use, the homogenate was diluted to a protein concentration of 0.6 mg/mL.

Peptide adsorption. Due to adsorption of EntF* to different kinds of plastic, all plastic tubes and containers were coated before use with a BSA-based anti-adsorption solution.

Metabolization kinetics. 500 μL of homogenate and 400 μL of KH buffer were mixed, together with 100 μL of KH buffer (blank) or 1 mg/mL EntF* peptide solution (test), all equilibrated and incubated at 37° C. After 0, 5, 10, 30, 60, 120 and 180 minutes, 100 μL aliquots were taken and immediately mixed with 100 μL of 1% V/V trifluoroacetic acid solution in water, heated for 5 min at 95° C., and cooled for 30 min in an ice-bath. After centrifugation at 16,000 g for 30 min at 5° C., supernatants were analysed by LC₁-MS₁ for EntF* quantification.

Intestinal permeability. Caco-2 cells were seeded on Transwell polycarbonate membrane filters (0.4 μm pore size) (Corning, Germany) at a density of 2.6×10⁵ cells/cm² and the permeability study performed as described by Hubatsch et al.²⁶. Cells were filled with Hank's Balanced Salt Solution (HBSS) and the TER values measured before and after the experiment. Peptide solution (1 μM) was added to the apical chamber and 300 μL aliquots taken after 30, 60, 90 and 120 min of incubation. Samples were analysed using LC₁-MS₁. Linear curve fitting was used to calculate the apparent permeability coefficient (Pₐₚₚ).

Standard protein BLAST. The amino acid sequence of the EntF* peptide was blasted against the NCBI non-redundant (nr) database by Basic Local Alignment Search Tool protein (BLASTp). This blast search was performed with the organism limited to bacteria (taxid:2). Only alignment hits with a 100% coverage and 100% identity were retained.

PCR on E. faecium strains. E. faecium was grown on MRS agar. DNA was extracted using alkaline lysis after which the EntF* fragment was amplified using 2× Biomix (Bioline, Belgium) in a Mastercycler PCR system (Eppendorf, Belgium). Each reaction was performed in a 10 μL total reaction mixture using 1 μL of the DNA sample and 0.5 μM final primer concentration (EntF*-PCR primers). The PCR conditions used: 1 cycle of 95° C. for 5 min, followed by 30 cycles of 94° C. for 30 sec, 55° C. for 30 sec and 72° C. for 1 min. Final elongation was performed at 72° C. for 10 min, after which the PCR product was hold at 4° C. The PCR amplification products were visualized on 1.5% agarose gel.

| | Forward/reverse | | SEQ ID |
|---|---|---|---|
| Target | primer | Sequence | NO: |
| EntF* | Forward | ACCTCAAGGAAAACCGGCATCA | 6 |
| | Reverse | TCAAGAGATCCCCTCCTGATTT | 7 |
| EntF | Forward | TGCTAAACAATGTTCAGATCAAGAGC | 8 |
| | Reverse | CCCACGAACATTCTTAATAGATAAGC | 9 |

Sample collection and preservation. Mice (C57BL/6) were euthanized by cervical dislocation and the blood collected. After standing for 30 min on ice, blood was centrifuged at 1,000 g for 10 min (room temperature). The supernatant (serum) was then transferred and stored at −35° C. until use.

After defaecation, two droppings of faeces were immediately collected and put in liquid nitrogen for max. 1 h. The samples were then stored at −80° C. until use.

Sample preparation. 50 μL of mice serum was mixed with 150 μL of 0.5% formic acid in acetonitrile. After sonication for 5 min and vortexing for 5 sec, the mixture was heated for 30 sec at 100° C. The solution was again vortexed and centrifuged for 20 min at 20,000 g (4° C.). The supernatant was then further purified using solid phase extraction (SPE) on HyperSep C₁₈ plates (Thermo Fisher Scientific, Belgium), which were previously conditioned with acetonitrile and equilibrated with 75% acetonitrile in water, containing 0.375% formic acid. After loading 150 μL of the samples, 120 μL eluent was collected and the organic solvents evaporated using nitrogen for 5 minutes. The resulting solutions were then further diluted with 30 μL of BSA-based anti-adsorption solution, followed by LC₁-MS₁ analysis.

LC₁-MS₁ analysis. EntF* was detected and quantified on a Waters Acquity UPLC H-class system, connected to a Waters Xevo™ TQ-S triple quadrupole mass spectrometer with electrospray ionization (operated in positive ionization mode). Autosampler tray and column oven were thermostated at 10° C.±5° C. and 60° C.±5° C., respectively. Chromatographic separation was achieved on a Waters Acquity® UPLC BEH Peptide C₁₈ column (300 Å, 1.7 μm, 2.1 mm×100 mm). The mobile phases consisted of 93:2:5 water:acetonitrile:DMSO (V/V) containing 0.1% formic acid (i.e. mobile phase A) and 2:93:5 water:acetonitrile:DMSO (V/V) containing 0.1% formic acid (i.e. mobile phase B), and the flow rate was set to 0.5 mL/min. From the samples, a 10 mL aliquot was injected. The gradient program started with 80% of mobile phase A for 1 minute, followed by a linear gradient to 40% of mobile phase A for 3.5 minutes. Gradient was then changed to 14.2% mobile phase A at 5 min, followed by a 1 min equilibration, before starting conditions were applied. EntF* showed retention at 4.25-4.45 min.

An optimised capillary voltage of 3.00 kV, a cone voltage of 20.00 V and a source offset of 50.0 V was used. Acquisition was done in the multiple reaction monitoring (MRM) mode. The selected precursor ion for EntF* was m/z 865.7 with two selected product ions at m/z 202.08 (36 V) as quantifier and m/z 315.17 (31 V) as qualifier.

A sample was considered positive for the presence of EntF* when following criteria were met: correct retention time, quantifier/qualifier peak area ratio's between 2.0 and 4.0, both quantifier (b2 fragment) and qualifier (b3 fragment) with a signal-to-noise ratio above 3.0 and a concentration above the LOQ of 100 pM.

LC₁-MS₂ analysis. Chromatographic separation was achieved on a Waters Acquity® UPLC HSS T3 Column (100 Å, 1.8 μm, 2.1 mm×100 mm), with detection using the Waters SYNAPT G2-Si High Definition Mass Spectrometry with electrospray ionization (operated in the positive ionization mode). Gradient composition and UPLC-MS settings were the same as with the LC₁-MS₁ method; a TOF-MS/MS mode was applied with a fixed mass on the quadrupole of 865.157, a fixed trap collision energy of 30 V and an acquired MS/MS over the range of 100-1450 m/z (scan time 1 second). EntF* retention was observed between 3.40-3.50 min. When at least four daughter ions (m/z±0.05) of EntF* were detected at the expected retention time and at least three of the most abundant isotope parent peaks (m/z±0.05) were detected, the sample was considered to contain the EntF* peptide.

LC₁-MS₃ analysis. While the UPLC separation system was the same as with the LC₁-MS₁ method, the third detection system consisted of a Thermo Fisher Q Exactive™ Hybrid Quadrupole-Orbitrap Mass Spectrometer. The mass spectrometer was operated using a heated electrospray ionization source with the following setting: capillary temperature set at 300° C., S-Lens RF level set at 50, spray voltage set at 3.00 kV and auxiliary gas flow set at 20.

A full MS/MS mode was applied with a fixed mass on the quadrupole of 865.157, a fixed trap collision energy of 30 V and 35 V and an acquired MS/MS over the range of 100-1800 m/z. EntF* retention was observed at 4.13-4.16 min. When at least two daughter ions (m/z±0.005) of EntF* were detected at the expected retention time and at least four of the most abundant isotope parent peaks (m/z±0.005) were detected, the sample was considered to be positive for the presence of EntF*.

LC₂-MS₁ analysis. Chromatographic separation was achieved on a Waters Acquity® UPLC BEH Amide Column (130 Å, 1.7 μm, 2.1 mm×100 mm). Mobile phase composition, sample volume, flow rate and MS settings were the same as described for the LC₁-MS₁ method. The gradient program started with 10% of mobile phase A for 2 minutes, followed by a linear gradient to 40% of mobile phase A for 3.0 minutes. Gradient was then changed to 85% mobile phase A at 6 min, followed by a 1 min equilibration, before starting conditions were applied. EntF* showed retention at 4.85-4.95 min. A sample was considered positive for the presence of EntF* when following criteria were met: correct retention time, both daughter fragments (i.e. b2 and b3) with a signal-to-noise ratio above 3.0 and quantifier/qualifier peak area ratio's between 2.0 and 4.0.

DNA extraction of faeces. To 20-40 mg faeces, 500 mg of unwashed glass beads, 0.5 mL CTAB buffer (hexadecyltrimethylammonium bromide 5% (w/v), 0.35 M NaCl, 120 mM K₂HPO₄) and 0.5 mL phenol-chloroform-isoamyl alcohol mixture (25:24:1) were added. The mixture was homogenized two times for 1.5 min at 22.5 Hz using a TissueLyser II (Qiagen, Belgium). The mixture was centrifuged for 10 minutes at 8,000 rpm and 300 μL of the supernatant was transferred to a new Eppendorf tube. For a second time, 0.25 mL of CTAB buffer was added to the original DNA sample, which was again homogenized in the TissueLyser and centrifuged for 10 minutes at 8,000 rpm. Of this supernatant, 300 μL was added to the first 300 μL supernatant. The phenol was removed by adding an equal volume of chloroform-isoamyl alcohol (24:1) followed by centrifugation at 16,000 g for 10 sec. The aqueous phase was transferred to a new tube. Nucleic acids were precipitated with 2 volumes PEG-6000 solution (polyethyleenglycol 30% (w/v), 1.6 M NaCl) for 2 h at room temperature. The pellet was obtained by centrifugation at 13,000 g for 20 min and washed with 1 mL of ice-cold 70% (v/v) ethanol. After centrifugation at 13,000 g for 20 min, the pellet was dried and resuspended in 50 μL de-ionized water. The quality and the concentration of the DNA was examined spectrophotometrically.

qPCR on faeces. qPCR was performed using SYBR-green 2× master mix in a Bio-Rad CFX-384 system. Each reaction was done in sixfold in a 12 μL total reaction mixture using 2 μL of the DNA sample and 0.5 μM final qPCR primer concentration. The qPCR conditions used: 1 cycle of 95° C. for 10 min, followed by 40 cycles of 95° C. for 30 sec, 60° C. for 30 sec, and stepwise increase of the temperature from 65° to 95° C. (at 10 sec/0.5° C.). Melting curve data were analysed to confirm the specificity of the reaction. Samples with a specific melting peaks were discarded from further analyses. After purification and determination of the DNA concentration, the concentration of the linear dsDNA standard was adjusted from 10⁷ to 10¹ copies (EntF*). The copy numbers of samples were determined by reading of the standard series with the Ct values of the samples. The standard curves were extrapolated for samples containing less than 10¹ copies. Because the Cq values of the EntF* qPCR analyses were around the limit of detection (LOD), with a notable amount of left-truncated data (data below LOD), a maximum likelihood (ML) approach was used to find the best estimation of mean and standard deviation for each sample.

Cell culture. Caco-2 and luciferase transfected HCT-8/E11 cells were grown in DMEM medium supplied with 10% foetal bovine serum (FBS) and 1% penicillin-streptomycin (10,000 U/mL) solution. The cells were cultured in an incubator set at 37° C. and 5% CO₂. When confluent, cells were detached using 0.25% trypsin-EDTA.

Orthotopic colorectal cancer mouse model. All in vivo experiments were performed according to the Ethical Committee principles of laboratory animal welfare and approved by our institute (Ghent University, Faculty of Medicine and Health Sciences, approval number ECD 17-90). Mice were maintained in a sterile environment with light, humidity and temperature control (light-dark cycle with light from 7:00 h to 17:00 h, temperature 21-25° C. and humidity 45-65%). Before the experiment, mice were allowed to acclimatize for a minimum of seven days.

Six-weeks old female athymic nude mice (Swiss nu/nu) were anesthetized and a small midline laparotomy executed to localize the caecum. The caecum was then gently exteriorized and luciferase transfected HCT-8/E11 cells (1×10⁶ cells) in a volume of 20 μL serum-free DMEM medium with matrigel (1:1) injected into the caecal wall. Cells were previously treated with EntF* (10 nM, 100 nM or 1 μM), Phr0662 (100 nM) or with the vehicle (PBS) or positive control (Transforming Growth Factor α (TGFα), 0.1 μg mL⁻¹) solution for 5 days before they were implanted in the mice. The caecum was then carefully returned to the abdominal cavity and the laparotomy closed in two layers by sutures of PDS 6/0. Starting from the day after tumour cell injection, mice were daily treated by i.p. injection with vehicle (PBS, n=15), EntF* (10 nmol kg⁻¹, n=12; 100 nmol kg⁻¹, n=18; 1 μmol kg⁻¹, n=8), Phr0662 (100 nmol kg⁻¹, n=5) or positive control (Epidermal Growth Factor (EGF), 100 μg kg⁻¹, n=18) for 6 weeks. Once a week, mice were investigated for tumour growth and metastases using bioluminescent imaging with the IVIS Lumina II (Perkin Elmer, Belgium) after i.p. injection with 200 μL luciferin (150 mg kg⁻¹). After 6 weeks, mice were euthanized using cervical dislocation, followed by macroscopic evaluation of the liver, diaphragm, lungs, caecum, duodenum and peritoneum for the presence of tumour nodules. Liver and lung tissues were then fixed in formalin during 24 h and stored in 70% ethanol for max. 3 days before embedding in paraffin. Afterwards, a haematoxylin & eosin (H&E) staining was performed on 8 μm sections and 3 sections were visualized per mouse using microscopy. The slides of all tumour-bearing mice were scored by two blinded, independent investigators using a scoring system. In the case of a difference in scoring, the slide was scored again by a third blinded, independent investigator for consensus.

Daily peptide exposures were calculated after i.p. injection of 25 μL of a 100 μM EntF* solution into female Swiss nu/nu mice (n=14), followed by LC₁-MS₁ analyses of mice serum at different time points after injection. A distribution and early exponential phase (α, 0-30 min), followed by a terminal elimination phase (β, 30-180 min) could be distinguished. The exposure was determined for 24 hours (x) as follows: Exposure (nM×min)=∫₀³⁰Ae^{-αt}+∫₃₀^{x}Be^{-βt}.

Western Blot analyses. 1×10⁶ luciferase transfected HCT-8/E11 cells were seeded in each well of a 6-well plate. 24 hours post-seeding, cells were treated with EntF* and its synthesised alanine-derived analogues (100 nM) or placebo, or with Nef-M1/QSP76S1A (10 μM) or mixtures (10M Nef-M1/QSP76S1A with 5 μM EntF*) to demonstrate one of the targets, i.e. CXCR4. After 24 hours, cells were detached from the surface and lysed with Thermo Scientific RIPA-buffer. The protein concentration was then determined using the modified Lowry protein assay kit, according to the manufacturer's instructions. All samples were diluted to the same concentration (i.e. 4 μg/μL) using water and diluted 1:1 using 2× Laemmli buffer. Next, the samples were boiled for 5 min at 95° C. for denaturation, after which centrifugation for 5 min at 16,000 g was performed; the supernatant was then used for Western blot analyses. Therefore, proteins (20 μg) were separated using a Bio-Rad Any kD gel (SDS-PAGE) and transferred to a PVDF membrane. Before the membranes were incubated with antibodies, non-specific binding sites were blocked using 5% skimmed milk solution (1 hour). Western blot was performed using an anti-E-cadherin (1/1000) antibody and incubated overnight at 4° C. Signal intensity was normalized against the total protein content in the lanes. Anti-rabbit-HRP antibody was used for detection (1/2000) (1 hour). Finally, the substrate (5 min) was added and the results were analyzed using the Bio-Rad ChemiDoc EZ imager and Image Lab software. TBS buffer with 0.05% Tween 20 was used for washing between the different steps.

Sample collection and preservation of human plasma. Blood was collected in a conventional EDTA tube and centrifuged at 3,000 g for 10 min (room temperature). The supernatant (plasma) was then transferred and immediately processed.

Sample preparation of human plasma. 900 μL of human plasma was mixed with 2.1 mL of 2% hydrochloric acid in a h (97/3) acetonitrile:DMSO solution. After sonication and vortexing for 1 min, the mixture was heated for 1 minute at 100° C. The solution was again vortexed and centrifuged for 1 min at 3,000 g. Subsequently, the supernatant (2 mL) was lyophilized and dissolved in 970 μL acetonitrile containing 0.1% formic acid. This solution was then further purified using AMIDE solid phase extraction (SPE). After the SPE column is conditioned with (90/10/0.1) water:acetonitrile:NH₄OH and equilibrated with (97/3/0.1) acetonitrile:DMSO:Formic acid, the sample is loaded and eluted with a (75/20/5) water:acetonitrile:DMSO:Formic acid solution in a BSA-based anti-adsorption diluent coated vial. The resulting solution was analysed using LC-MS analysis.

Statistical analyses. The Kolmogorov-Smirnov test was used to assess if data obtained were normally distributed. For sample sizes of n<10, non-parametric tests (Mann-Whitney U test) were performed directly. Slope comparison was based on linear regression analysis. Bootstrapped medians and Hedges G-values were used to calculate the effect size.

### Results

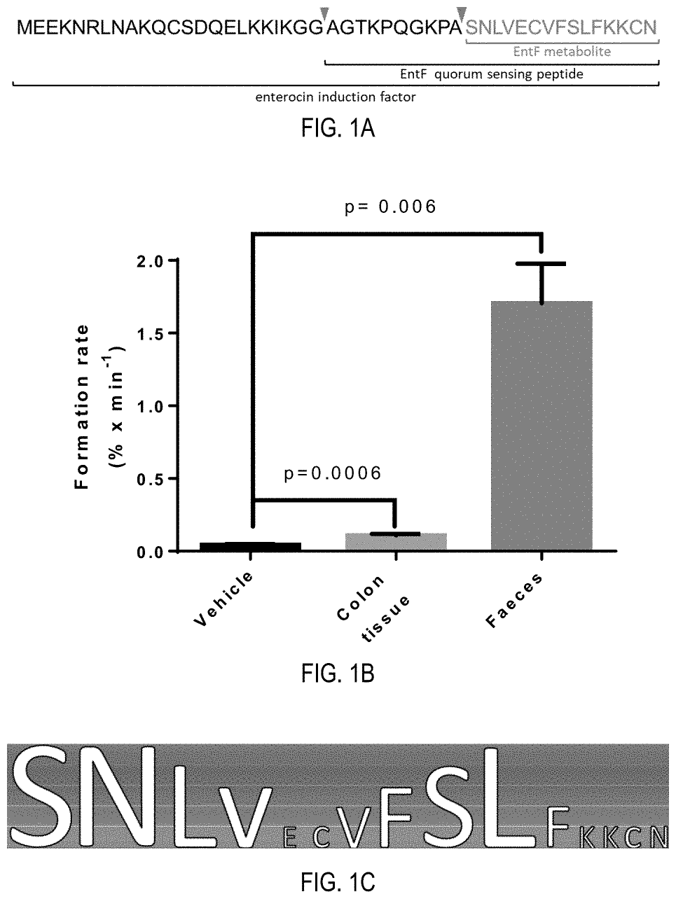

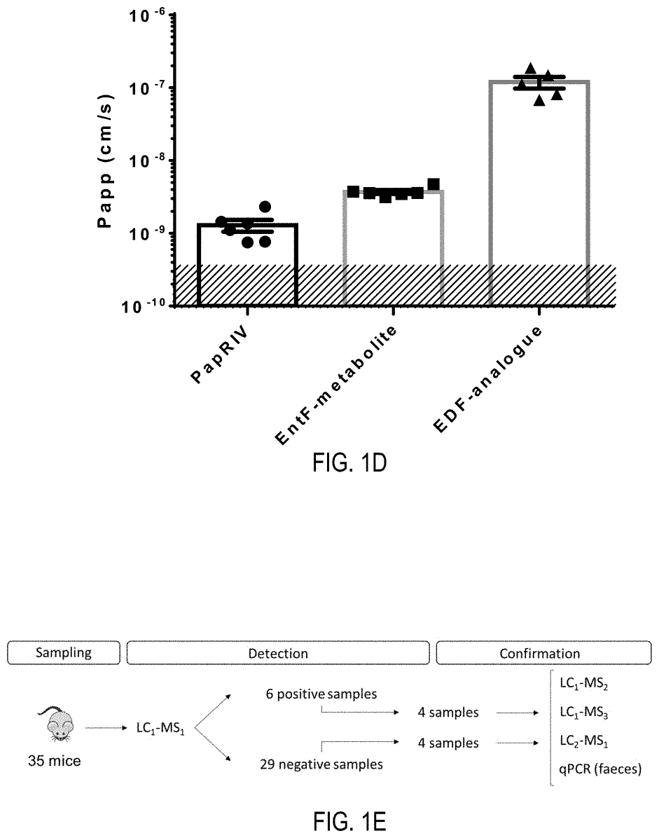

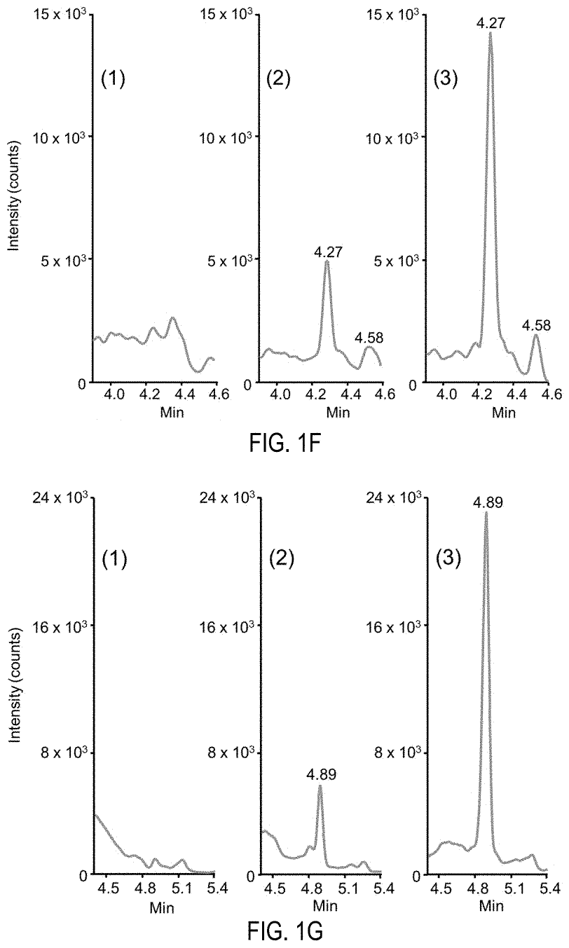

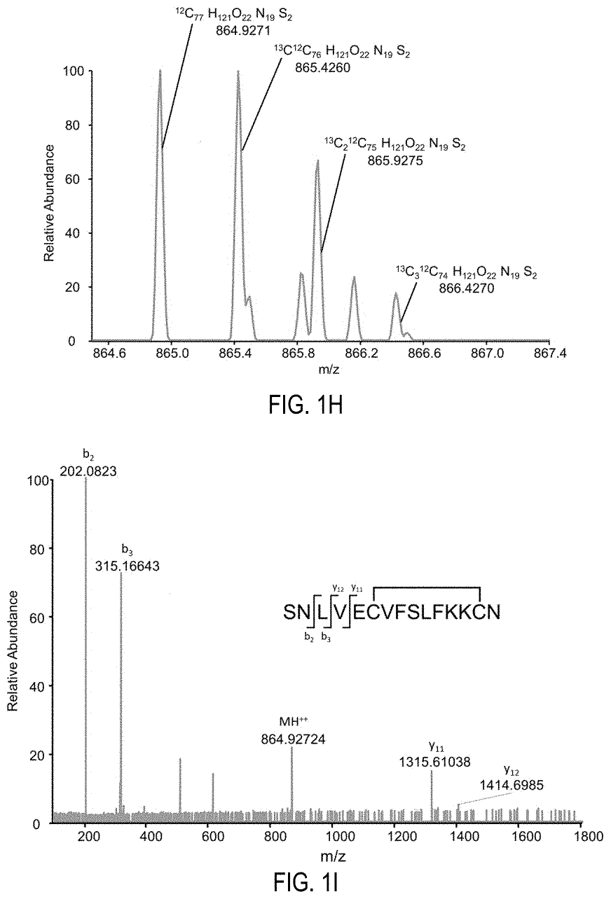

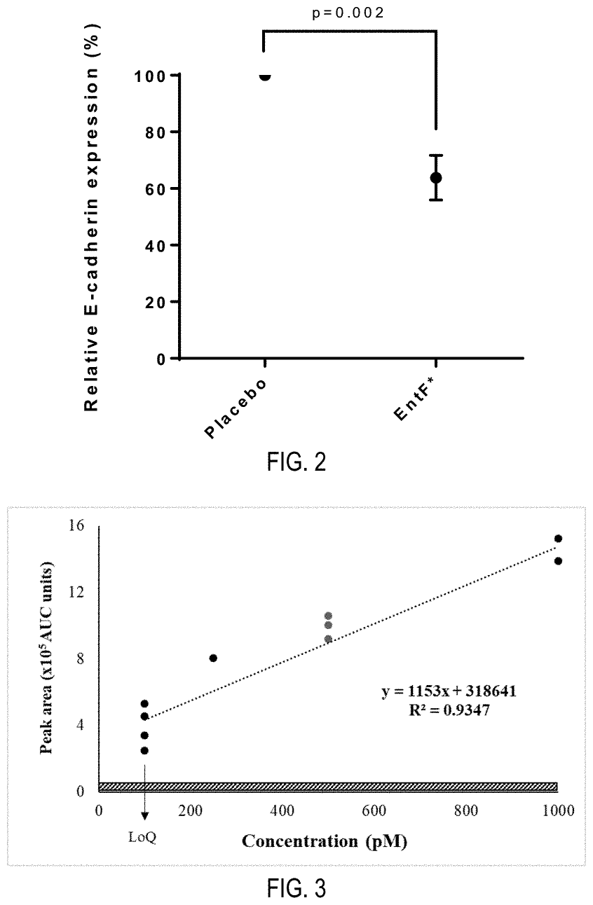

Quorum sensing peptides are traditionally regarded as intra- and inter-bacterial communication molecules, but given their wide structural variety and co-evolution, we anticipate that these molecules may also interact with the host. Up till now, however, quorum sensing peptides have not yet been unambiguously demonstrated to be present in biofluids. Only an indirect indication of the in vivo presence of an unidentified quorum sensing peptide was described in the stool of patients suffering from a Clostridium difficile infection¹². We have previously focussed on Enterococcus faecium, one of the most abundant species in the human intestinal microbiota, which synthesizes the enterocin induction factor, i.e. the EntF quorum sensing peptide (AGTKPQGKPASNLVECVFSLFKKCN, SEQ ID NO:2, FIG. 1A). This peptide serves as a communication signal, regulating the production of enterocin A and B toxins, which are produced to inhibit the growth of similar or closely related bacterial strains¹³⁻¹⁷. Standard protein BLAST searches thereby indicate that EntF-producing E. faecium strains indeed are present in different human faeces samples (Table 1).

TABLE 1
Bacterial strains found in human faeces samples with
indicated presence of the EntF gene in the genome.
Using BLASTp searches, the isolation source
was investigated for the resulting E. faecium strains, with
different strains found in human faeces samples.
EntF producing strains found in human faeces

| | |
|---|---|
| E. faecium LIM714, | E. faecium 2014-VREF-41, |
| E. faecium VRE-1402237, | E. faecium 2014-VREF-63, |
| E. faecium U-1313438, | E. faecium 2014-VREF-26, |
| E. faecium E1071, | E. faecium F1129F 09, |
| E. faecium ERV165, | E. faecium F1213D 01, |
| E. faecium GMD5E, | E. faecium 24, |
| E. faecium GMD4E, | E. faecium Hp_24-3, |
| E. faecium GMD3E, | E. faecium Hp_5-10, |
| E. faecium GMD2E, | E. faecium Hp_6-10, |
| E. faecium GMD1E, | E. faecium Hp_5-7, |
| E. faecium 6E6, | E. faecium 8_Efcm_HA-DE, |
| E. faecium MXVK29, | E. faecium 11_Efcm_HA-DE, |
| E. faecium S-1001508, | E. faecium FDAARGOS_326, |
| E. faecium VRE-1402513, | E. faecium 4278, |
| E. faecium VRE-1402563, | E. faecium 1584, |
| E. faecium VRE-1406033, | E. faecium IHC105, |
| E. faecium VRE-1504220, | E. faecium IHC116, |
| E. faecium LMG 8148, | E. faecium IHC106, |
| E. faecium Isolate 30, | E. faecium IHC113, |
| E. faecium VRE16, | E. faecium IHC115, |
| E. faecium XH877, | E. faecium IHC130, |
| E. faecium HMSC063H10, | E. faecium IHC117, |
| E. faecium HMSC069A01, | E. faecium IHC121, |
| E. faecium HMSC056C08, | E. faecium IHC118, |
| E. faecium 97-7_S6, | E. faecium IHC127, |
| E. faecium LIM1546, | E. faecium IHC108, |
| E. faecium LIM1547, | E. faecium IHC120, |
| E. faecium LIM1759, | E. faecium IHC123 |

On the other hand, our experimental data indicated that not all E. faecium strains have this EntF gene in their genome (Table 2), indicative for the inter-individual variation in the (gut) microbial community.

TABLE 2
Presence of the EntF gene in different E. faecium strains. Different, commercially
available strains of E. faecium were investigated for the presence of the EntF gene using
PCR. For some strains, the EntF gene was confirmed, while this was absent in other strains.

| Bacterial strain | Origin | EntF gene present? |
|---|---|---|
| E. faecium LMG 20720 | Human faeces | Yes |
| E. faecium LMG 23236 | Human faeces (healthy) | Yes |
| E. faecium LMG 15710 | Human faeces (diarrhea) | No |
| E. faecium ATCC 8459 | Dairy product (cheese) | Yes |
| E. faecium T110 | Unspecified | Yes |
| E. faecium NCIMB 10415 (SF68) | Unspecified | No |

We demonstrated that metabolization of EntF in faeces and colonic tissue homogenate yielded a 15-mer peptide EntF* (SNLVECVFSLFKKCN, SEQ ID NO:1) (FIG. 1). This quorum sensing peptide metabolite was previously found by our group to promote angiogenesis and tumour cell invasion in in vitro experiments using HCT-8 CRC cells¹⁸. Effects on E-cadherin expression of this EntF* peptide and some alanine-derived analogues were observed, which is linked to epithelial-mesenchymal transition (EMT) of the cancer cells (FIG. 1C). Moreover, similar to other quorum sensing peptides¹⁹, EntF* is able to cross the intestinal barrier in vitro, using a CaCo-2 monolayer permeability assay (FIG. 1D). These in vitro studies thus indicated that EntF* can be present in the blood circulation of the host, after degradation of EntF in the colon or faeces and subsequent intestinal absorption. To unambiguously demonstrate its in vivo presence, a bioanalytical method using RP-UPLC-TQ-MS in MRM-modus was developed and optimized, aiming to avoid carry-over and adsorption, as well as to maximize the selectivity and sensitivity. Critical methodological aspects to achieve these goals were: (1) a suitable sample preparation method using a novel bovine serum albumin (BSA)-based anti-adsorption solution and the combination of solvent/acid/heat sample treatment followed by SPE, (2) a judicious choice of the starting mobile phase and gradient slope in the ultra-high-performance liquid chromatography (UPLC) method, and (3) appropriate mass spectrometry (MS) detection settings, including the selection of quantifier (b₂: m/z=202.08) and qualifier (b₃: m/z=315.17). The method was suitably verified and found to be appropriate for its purpose.

### Bio-Analytics for EntF* in Serum/Plasma

Mice serum is mixed (1/4) with of 0.5% formic acid in acetonitrile. After sonication and vortexing, the mixture is boiled. The solution is again vortexed and centrifuged. The supernatant is then further purified using solid phase extraction (SPE). After loading the samples, the eluent is collected in a BSA-based anti-adsorption diluent coated vial and the organic solvents evaporated using nitrogen. The resulting solutions are then further diluted with BSA-based anti-adsorption solution, followed by LC-MS analysis.

### Sample Preparation for Peptidomics in Plasma

Plasma is mixed (3/10) with (94/3/3) acetonitrile:DMSO:Formic acid. After sonication and vortexing, the mixture was boiled and subsequently put on ice. The solution was centrifuged and the supernatant is diluted (1/9) with (97/3/0.1) acetonitrile:DMSO:Formic acid. The supernatant is then further purified using AMIDE solid phase extraction (SPE). After the SPE column is conditioned with (90/10/0.1) water:acetonitrile:NH4OH and equilibrated with (97/3/0.1) acetonitrile:DMSO:Formic acid, the sample is loaded and eluted with a (75/20/5) water:acetonitrile:DMSO:Formic acid solution in a BSA-based anti-adsorption diluent coated vial. The resulting solution get analysed using LC-MS analysis.

### Sample Preparation for Peptidomics in Feces

Feces are mixed (1/1) with acidified water. After sonication and vortexing, the solution is diluted (1/1) with (97/3/0.1) acetonitrile:DMSO:Formic acid. The solution is again vortexed and sonicated and is subsequently boiled and centrifuged. The supernatant is diluted (1/9) with (97/3/0.1) acetonitrile:DMSO:Formic acid. The supernatant is then further purified using AMIDE solid phase extraction (SPE). After the SPE column is conditioned with (90/10/0.1) water:acetonitrile:NH4OH and equilibrated with (97/3/0.1) acetonitrile:DMSO:Formic acid, the sample is loaded and eluted with a (75/20/5) water:acetonitrile:DMSO:Formic acid solution in a BSA-based anti-adsorption diluent coated vial. The resulting solution get analysed using LC-MS analysis.

### Chromatographic Method

Chromatographic separation was achieved on C18 reverse phase column. The mobile phases consisted of 93:2:5 water:acetonitrile:DMSO (V/V) acidified with formic acid (i.e. mobile phase A) and 2:93:5 water:acetonitrile:DMSO (V/V) acidified with formic acid (i.e. mobile phase B), and the flow rate was set to 0.5 mL/min. From the samples, a 10 μL aliquot was injected. The gradient program started with 80% of mobile phase A, followed by a linear gradient to 40% of mobile phase A. Gradient was then changed to 14.2% mobile phase A, followed by an equilibration, before starting conditions were applied. Serum samples of 35 healthy mice (C57BL/6 mice, aged 5-18 months) were collected and analysed for the presence of EntF* (FIG. 1E). For six mice, the presence of EntF* was observed in their serum above the limit of quantification (LOQ) of 100 pM, with an overall estimated mean value of 305 pM (standard error of the mean, s.e.m.=138 pM; n=35) (FIG. 1F) (Table 3).

TABLE 3
Concentration ofEntF* measured in mice serum using
LC1-MS1 method. Out of the 35 serum samples,
6 tested positive for the presence of EntF*, indicated in bold.
Four positive and four negative samples, indicated
in italic, were used for the confirmatory experiments. Taking
all 35 results into consideration, with <LOQ set equal to zero,
then the average ± s.e.m. is 329 pM ± 150 pM (n = 35).
If only the six positive samples are considered, then the
average ± s.e.m. is 1.91 nM ± 0.54 nM (n = 6).

| | | Measured |
|---|---|---|
| Sample | ID | concentration (pM) |
| 1 | 20180827S1 | <LOQ |
| 2 | 20180827S2 | <LOQ |
| 3 | 20180827S3 | <LOQ |
| 4 | 20180827S5 | <LOQ |
| 5 | 20180827S6 | <LOQ |
| 6 | 20180827S7 | <LOQ |
| 7 | 20180827S8 | <LOQ |
| 8 | 20180827S9 | <LOQ |
| 9 | 20180827S10 | <LOQ |
| 10 | 20180829S2 | <LOQ |
| 11 | 20180829S3 | <LOQ |
| 12 | 20180829S4 | <LOQ |
| 13 | 20180829S5 | <LOQ |
| 14 | 20180829S6 | <LOQ |
| 15 | 20180829S7 | <LOQ |
| 16 | 20180829S8 | <LOQ |
| 17 | 20180829S9 | <LOQ |
| 18 | 20181011S1 | <LOQ |
| 19 | 20181011S2 | <LOQ |
| 20 | 20181011S3 | <LOQ |
| 21 | 20181011S4 | 389 |
| 22 | 20181011S5 | <LOQ |
| 23 | 20181011S6 | <LOQ |
| [symbol] | [symbol] | [symbol] |
| 25 | 20181011S8 | <LOQ |
| [symbol] | [symbol] | [symbol] |
| 27 | 20181011S10 | <LOQ |
| 28 | 20181011S11 | <LOQ |
| [symbol] | [symbol] | [symbol] |
| 30 | 20181011S13 | <LOQ |
| 31 | 20181011S14 | <LOQ |
| 32 | 20181011S15 | <LOQ |
| [symbol] | [symbol] | [symbol] |
| 34 | 20181011S17 | <LOQ |
| 35 | 20181011S18 | 200 |

Following these findings, further proof was obtained by subjecting a selected set of samples to three additional methods: HILIC-UPLC-TQ-MS (FIG. 1G) as an orthogonal separation system, and RP-UPLC-QOrbitrap-MS and RP-UPLC-QTOF-MS as high-resolution mass spectrometers. Serum samples of eight mice (four positive and four negative samples (i. e. above and below the LOQ, respectively, based on the RP-UPLC-TQ-MS findings)) were analysed using these additional methods (FIGS. 1G, 1H, and 1I). The presence and identity of EntF* was confirmed using the isotopic distribution of the doubly charged precursor ion (FIG. 1H) and the fragment ions y₁₁ (m/z=1315.61) and y₁₂ (m/z=1414.69) (FIG. 1I) in the four positive serum samples. Finally, quantitative real-time PCR analysis on the associated faeces samples was performed to demonstrate the existence of EntF*-containing E. faecium DNA copies (Table 4): EntF* DNA copies were indeed observed in all four positive samples. In addition, standard protein BLAST searches indicated no endogenous presence of the EntF* peptide sequence in the murine genome, again demonstrating the microbial origin of the in vivo found peptide.

TABLE 4
qPCR detection of EntF*. DNA was extracted out of
20-40 mg of faeces and qpCR was performed in 6-fold
using indicated inner primers (green). Standard curves were
made using the amplicon limited by indicated outer primers
(purple). EntF* DNA copies were observed in all 4 LC-MS
positive samples (i.e. 20181011S7, 20181011S9,
20181011S12, 20181011S16); no EntF* copies
could be detected in sample 20181011S10.

| | Number of EntF* |
|---|---|
| Sample ID | copies/g faeces |
| 20181011S7 | 588 |
| 20181011S8 | 612 |
| 20181011S9 | 3060 |
| 20181011S10 | — |
| 20181011S11 | 39 |
| 20181011S12 | 64 |
| 20181011S13 | 375 |
| 20181011S16 | 1858 |

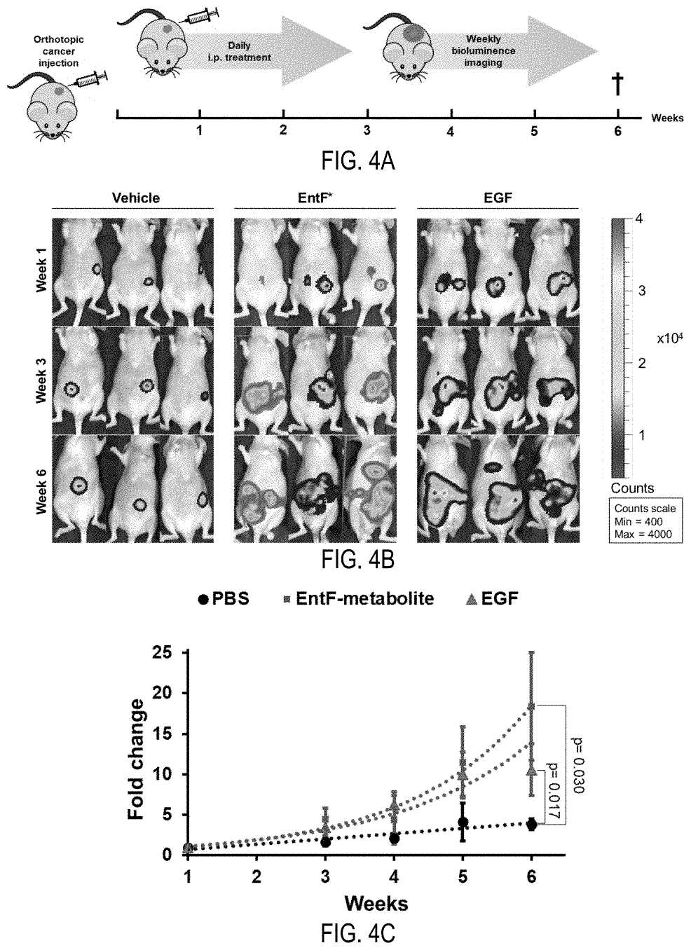

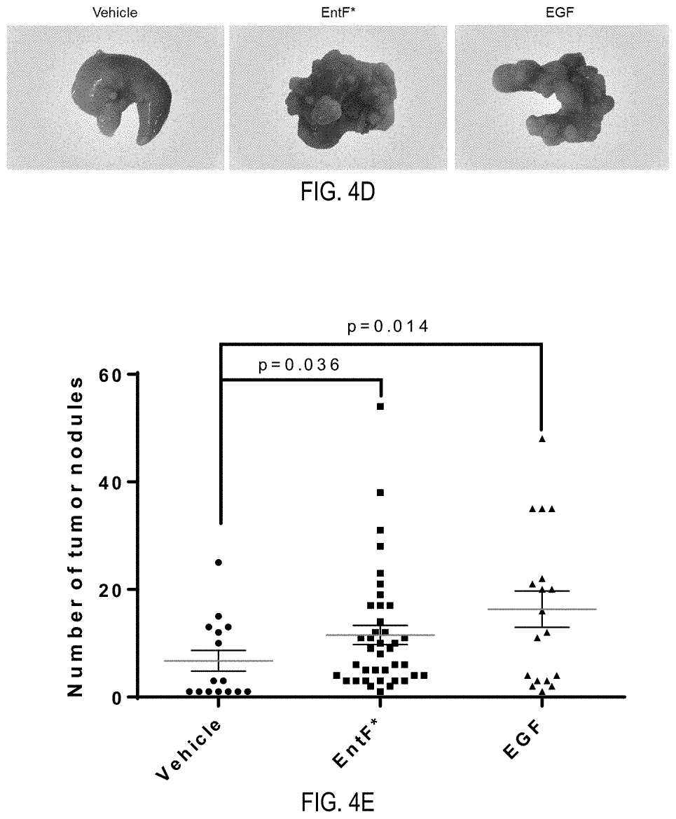

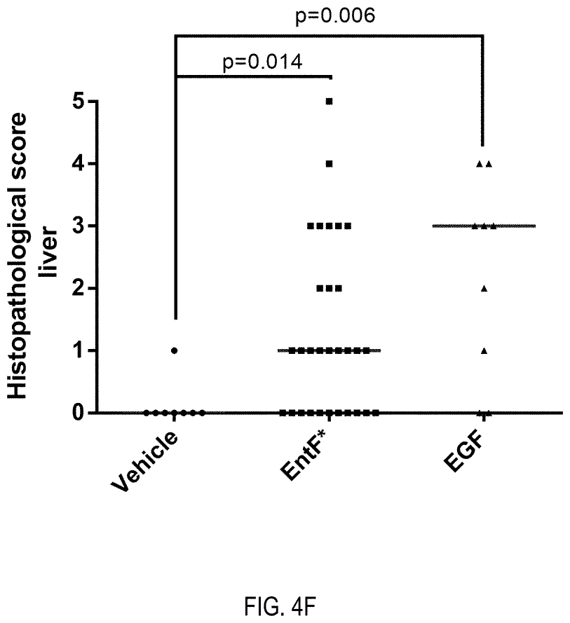

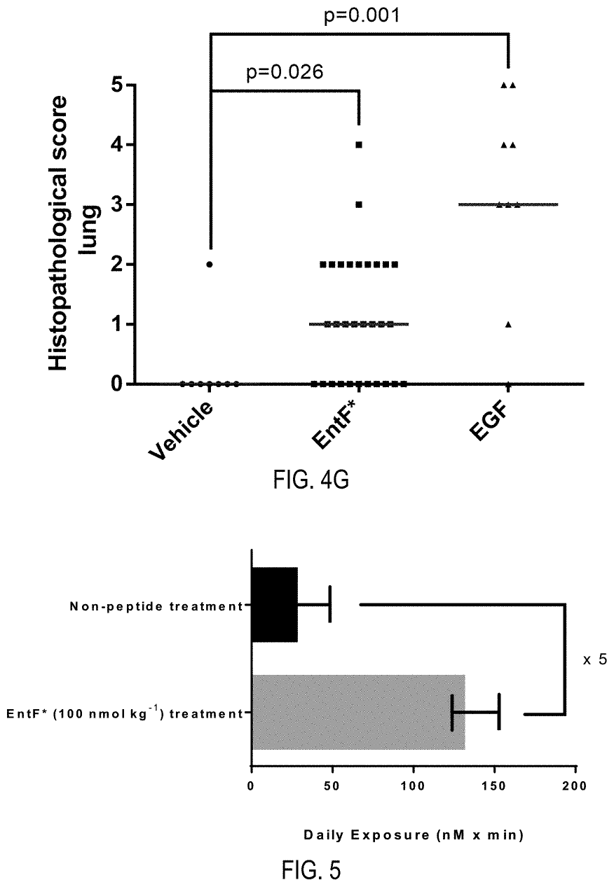

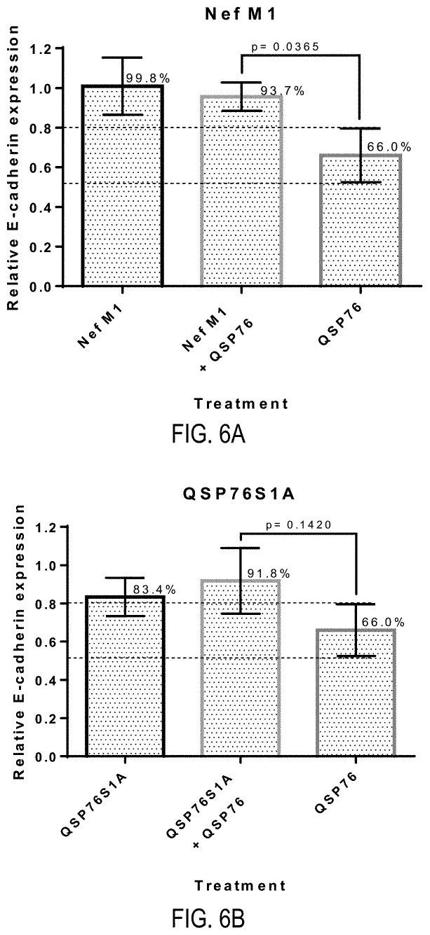

Having demonstrated the presence of EntF* in vivo both directly and indirectly, we evaluated its in vivo metastasis-promoting activities using an orthotopic colorectal cancer mouse model^{20,21}. Before the luciferase transfected HCT-8 cells were implanted into the wall of the caecum of the mice, cells were treated daily for five days with EntF* (100 nM), phosphate-buffered saline (PBS) vehicle or Transforming Growth Factor α (TGFα, positive control) (0.1 μg mL⁻¹). On the sixth day, 5-weeks-old female Swiss nu/nu mice were orthotopically injected with 1×10⁶ luciferase transfected colorectal cancer cells, followed by a once-daily i.p. treatment of EntF* (100 nmol kg⁻¹), PBS vehicle or Epidermal Growth Factor (EGF, positive control) (100 μg kg⁻¹) (FIG. 4A). Daily injections of 100 nmol kg⁻¹ EntF* thereby gave daily peptide exposures which were five times higher than the endogenous (natural) exposure in those mice. Bioluminescent imaging was performed weekly to monitor the tumour growth (FIG. 4B). During the course of 6 weeks, EntF* caused a statistically significant relative increase in luciferase activity compared to vehicle (p=0.017), which was even not significantly different from the positive control EGF (p=0.319; FIG. 4C). Our results demonstrated, after 6 weeks treatment, an effect size of 128% increase in bioluminescence for EntF* compared to the placebo PBS, varying from −75%, a relative small negative association, to a 1494% increase, a substantial positive association (Table 5). For the positive control EGF, a median effect size of 316% was obtained, ranging from −145% to 850%. This was confirmed by the number of tumour nodules counted macroscopically on the caecum (FIG. 4D), which was again statistically significantly higher with EntF* in comparison to PBS (FIG. 4E), demonstrating the in vivo 3-fold increase in the number of nodules due to this quorum sensing peptide metabolite while EGF showed a 4.5-fold increase (Table 6).

TABLE 5
Effect size for bioluminescence in the orthotopic mouse model after 6 weeks
treatment. After 6 weeks treatment, an effect size of 128% increase in bioluminescence
for EntF* compared to the placebo PBS was observed, while for the positive control EGF,
a median effect size of 316% was obtained. When calculating the effect size according
to Hedges’ G values, a median to high effect was observed for both the EntF* and the
EGF treatment groups, compared to the placebo group.

| | Number of | Median | | Bootstrapped median | | Hedges’ g value |
|---|---|---|---|---|---|---|
| Treatment | mice | (95% CI) | | difference (95% CI) | | (vs PBS) |
| PBS | 12 | 314 | (247 to 471) | 0 | (0 to 0) | — |
| EntF* | 17 | 442 | (267 to 1829) | 128 | (−75 to 1494) | 0.6 |
| EGF | 16 | 630 | (194 to 1288) | 316 | (−145 to 850) | 0.7 |

TABLE 6
Effect size for the number of nodules on the caecum after 6 weeks treatment. After
6 weeks treatment, a 3-fold increase in the number of nodules on the caecum was
observed for EntF* compared to the placebo PBS, while for the positive control
EGF, a 4.5-fold increase was obtained. When calculating the effect size according
to Hedges’ G values, a median and high effect was observed for the EntF* and EGF
treatment groups, respectively, compared to the placebo group.

| | Number of | Median | | Bootstrapped median | | Hedges’ g value |
|---|---|---|---|---|---|---|
| Treatment | mice | (95% CI) | | difference (95% CI) | | (vs PBS) |
| PBS | 15 | 3 | (1 to 12) | 0 | (0 to 0) | — |
| EntF* | 39 | 9 | (5 to 11) | 5 | (−6 to 10) | 0.5 |
| EGF | 14 | 14 | (3 to 21) | 9 | (−6 to 20) | 0.8 |

Histopathological data further showed a significant higher number of tumours in both lungs and liver (FIGS. 4F and 4G). This is important, as the liver is the most common site of metastases from colorectal cancer: in clinical practice, up to half of all patients with colorectal cancer will develop hepatic metastases, with a median survival of only 8 months and a 5-year survival of less than 5%²²⁻²⁵. Remarkably, another quorum sensing peptide (i.e. Phr0662, ERNNT, synthesized by Bacillus species), which also demonstrated to promote in vitro cell invasion¹⁸, showed no in vivo metastasis-promoting effects.

Nef-M1 blocks the effect of QSP76 on the CXCR4 receptor by bringing the reduced relative E-cadherin expression from 66.0% (SEM=12.1%) caused by QSP76, back to 93.7% (SEM=7.0%). This significant normalisation in E-cadherin expression (one-sided student's t test: p=0.0365; very large Cohen's d effect size of 1.22), together with the similar E-cadherin expression of Nef-M1 (99.3%±13.5%) compared to PBS (100%), proves that Nef-M1 can serve as a competitive antagonist by blocking the binding of QSP76 on the CXCR4 receptor. QSP76S1A goes in competition with QSP76 on the CXCR4 receptor by bringing the reduced relative E-cadherin expression from 66.0% (SEM=12.1%) caused by QSP76, back to 91.8% (SEM=15.7%) (one-sided student's t test: p=0.1420; medium Cohen's d effect size of 0.77).

Collectively, our findings demonstrate that EntF*, a quorum sensing peptide metabolite, is present in vivo in biofluids of mice and promotes the metastasis of CRC in an orthotopic animal model, with a potency comparable to that of the well-established human colorectal cancer growth factor, EGF. Our findings indicate that quorum sensing peptides are an additional factor influencing microbiota-host interactions during CRC metastasis. This offers new possibilities in disease prevention, diagnosis and therapy by selective modulation of the gut microbiome as provided herein.

### Sample Preparation of Human Plasma.

Human plasma as well as feces samples of healthy volunteers were investigated for the presence of EntF*. An additional lyophilisation step was used in addition to the analytical methodology as provided herein, in order to further improve the detection limits, e.g. to 0.1 pM for plasma. In the majority of the feces samples, EntF* was found with concentration levels ranging between 20 to 800 pM, while in some plasma samples, levels of around 3 pM were found. These findings highlight further the human clinical relevance of our findings, and evidence that plasma and feces detection of EntF* is feasible, such as in the context of cancer metastasis risk monitoring.

### In Vivo Experiment

Western Blot analyses. 10⁶ HCT-8 (n=15), HCT-116 (n=12), CaCo-2 (n=6) and HT-29 (n=12) cells were seeded in each well of a 6-well plate. 24 hours post-seeding, cells were treated with EntF* (all cells with 1 μM), CXCL12 (10 ng/ml) or placebo. After 24 hours, cells were detached from the surface and lysed with Thermo Scientific RIPA-buffer. The protein concentration was then determined using the modified Lowry protein assay kit, according to the manufacturer's instructions. All samples were diluted to the same concentration (i.e. 4 μg/μL) using water and diluted 1:1 using 2× Laemmli buffer. Next, the samples were boiled for 5 min at 95° C. for denaturation, after which centrifugation for 5 min at 16,000 g was performed; the supernatant was then used for Western blot analyses. Therefore, proteins (20 μg) were separated using a Bio-Rad Any kD gel (SDS-PAGE) and transferred to a PVDF membrane. Before the membranes were incubated with antibodies, non-specific binding sites were blocked using 5% skimmed milk solution (1 hour). Western blot was performed using an anti-E-cadherin (1/1000) antibody and incubated overnight at 4° C. Signal intensity was normalized against the total protein content in the lanes. Anti-rabbit-HRP antibody was used for detection (1/2000) (1 hour). Finally, the substrate (5 min) was added and the results were analyzed using the Bio-Rad ChemiDoc EZ imager and Image Lab software. TBS buffer with 0.05% Tween 20 was used for washing between the different steps.

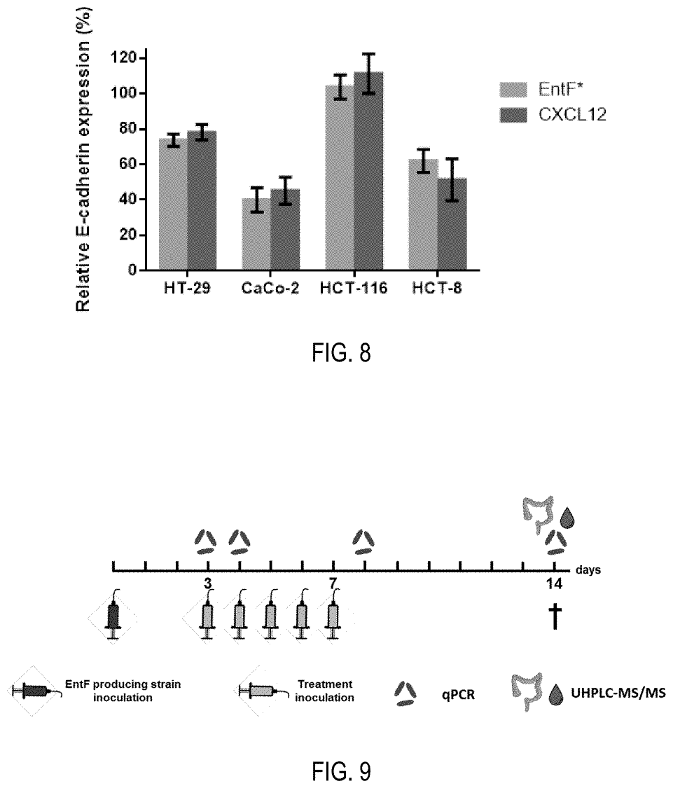

Result Western blot. Treatment of CXCR4 positive cell lines (HCT-8, HT-29 and CaCo-2) with EntF* results in a statistically significant (p<0.0001) decreased expression of E-cadherin (decrease of 26%, 60% and 38% respectively). Alternatively, when a CXCR4 negative cell line (HCT-116) is treated with EntF*, no decrease was observed (FIG. 8).

Gnotobiotic mouse model. Eight male gnotobiotic C57BL6 mice (housed randomly in three different cages) were treated once with a mixture of three EntF positive E. faecium (i.e. E. faecium LMG23236, T-110 and ATCC 8459) at a concentration of 10⁸ CFU/300 μL per strain. Three days after this pre-treatment, the mice are equally divided into two treatment groups, kept separately per group in separate cages. The following treatment groups were applied (each treatment applied during five consecutive days): (1) The placebo group was treated five consecutive days with the cell medium (BHI). (2) The test group received a mixture of three EntF negative E. faecium strains (10⁸ CFU) suspended in 300 μl of a BHI broth for five consecutive days (i.e E. faecium NCIMB 10415, W54 and LMG S-28935). After the five day treatment, the amount of EntF-producing strains in the different groups were monitored using qPCR for another week without treatment. The entire experiment was performed under gnotobiotic conditions. The faeces (2 droplets/day) of each mice was collected at day 3, 4, 8 and 14, immediately put in suitable, pre-labelled recipients, put in liquid nitrogen and stored at −80° C. Later, the faeces samples were analysed for DNA presence of EntF*. At day 14, the faecal droplets were collected, the mice were sacrificed and the blood and the whole colon (including the content) were obtained. Blood samples were immediately transferred to a 1.5 ml Eppendorf, put on ice for 10 to 30 min and centrifuged at 1,000 g for 10 min per group. The serum was stored at −80° C. and analysed for the presence of EntF* using UHPLC-MS². Colon contents were immediately transferred to a 15 ml tube, put on dry ice for 1 to 3 hours and stored at −80° C. The contents were removed and analysed for the presence of EntF using UHPLC-MS². An overview of the experiment is given in FIG. 9.

Sample preparation serum. 50 μL of mice serum was mixed with 150 μL of 0.5% formic acid in acetonitrile. After sonication for 5 min and vortexing for 5 sec, the mixture was heated for 30 sec at 100° C. The solution was again vortexed and centrifuged for 20 min at 20,000 g (4° C.). The supernatant was then further purified using solid phase extraction (SPE) on HyperSep C18 plates (Thermo Fisher Scientific, Belgium), which were previously conditioned with acetonitrile and equilibrated with 75% acetonitrile in water, containing 0.375% formic acid. After loading 150 μL of the samples, 120 μL eluent was collected and the organic solvents evaporated using nitrogen for 5 minutes. The resulting solutions were then further diluted with 30 μL of BSA-based anti-adsorption solution, followed by LC-MS analysis.

Sample preparation colon. For EntF analysis in colon content, 50 mg of colon content is suspended in 100 μL of a 2% hydrochloric acid solution which is vortexed and sonicated for 30 seconds. The suspension is again centrifuged for 1 min at 3,000 g and 50 μL of supernatant was heated for 1 min at 100° C. and cooled down for 1 min on ice. This solution was centrifuged again for 1 min at 10,000 g and too 30 μL of supernatant, 900 μL of a 3% DMSO solution in acetonitrile acidified by adding 0.1% formic acid was added (=equilibration solution). After a previously conditioning step with acetonitrile and an equilibration step with the equilibration solution, 900 μL of the diluted sample is loaded on a HILIC amide SPE MonoSpin column. The sample was eluted using a mixture of 75/20/5 (V/V/V) H₂O/acetonitrile/DMSO acidified by adding 0.1% formic acid, followed by LC-MS analysis.

LC-MS analysis. EntF and EntF* were detected and quantified on a Waters Acquity UPLC H-class system, connected to a Waters Xevo™ TQ-S triple quadrupole mass spectrometer with electrospray ionization (operated in positive ionization mode). Autosampler tray and column oven were thermostated at 10° C.±5° C. and 60° C.±5° C., respectively. Chromatographic separation was achieved on a Waters Acquity® UPLC BEH Peptide C18 column (300 Å, 1.7 μm, 2.1 mm×100 mm) for serum and colon content samples. The mobile phases consisted of 93:2:5 water:acetonitrile:DMSO (V/V/V) containing 0.1% formic acid (i.e. mobile phase A) and 2:93:5 water:acetonitrile:DMSO (V/V/V) containing 0.10% formic acid (i.e. mobile phase B), and the flow rate was set to 0.4 mL/min. From the samples, a 10 μL aliquot was injected. The gradient program started with 80% of mobile phase A for 30 sec, followed by a linear gradient to 40% of mobile phase A for 7 minutes. Gradient was then changed to 14.2% mobile phase A at 7.5 min, followed by a 30 sec equilibration, before starting conditions were applied. An optimised capillary voltage of 3.00 kV, a cone voltage of 20.00 V and a source offset of 50.0 V was used. Acquisition was done in the multiple reaction monitoring (MRM) mode. The selected precursor ion for EntF was m/z 667.10 with two selected product ions at m/z 949.39 (CE: 22 eV, Y₁₇ fragment) as quantifier and m/z 129.07 (CE: 30 eV, b₂ fragment) as qualifier. The selected precursor ion for EntF* was m/z 865.18 with two selected product ions at m/z 202.08 (CE: 36 eV, b₂ fragment) as quantifier and m/z 315.15 (CE: 31 eV, b₃ fragment) as qualifier.

Statistical analyses. To determine statistical difference in EntF*-copies between the placebo and the test group during the live biotherapeutic potential test of E. faecium, a two-way ANOVA was used. Significant differences (p<0.05; with treatments (column) and time (row) as the two factors; and H₀: the average parameter value for the different factor-levels are equal, and no interaction) between the treatments and time were evaluated. As a post-hoc analysis, a bonferroni's multiple comparison test (within each row, compare columns and within each column, compare rows) was applied. Graphpad was used to display the data, determine the statistical differences and calculate the 95% confidence interval mean difference between the test and placebo groups over time. The statistically significant differences of EntF concentrations in the colon and EntF* concentrations in serum were determined using a two-tailed unpaired t test (H₀: the average peptide content in the different groups are equal) in Graphpad.

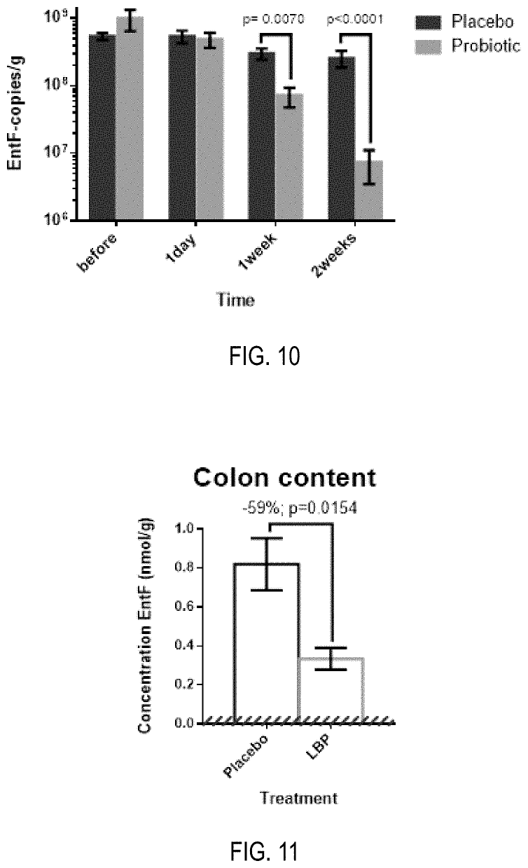

Result gnotobiotic mice experiment. After one day of treatment, a non-significant reduction of 10% EntF*-copies was determined in the test group compared to placebo. However, after five days of treatment (1 week in FIG. 10), a significant reduction of 80% (p=0.0069) EntF*-copies was determined in the test group compared to placebo. Additionally, even seven days post treatment (2 weeks in FIG. 10), the reduction in EntF*-copies continued with a significant decrease of 97% (p<0.0001) in EntF*-copies compared to placebo.

In the colon content of the placebo group, that did not receive any treatment, an average concentration of 820 pmol/g (SEM=134 pmol/g) EntF was determined. However, the level of EntF decreased significantly with 59%, to 335 pmol/g (SEM=56 pmol/g), when mice were treated with the test mixture. In FIG. 11, the difference in colon content concentrations between the groups is shown.

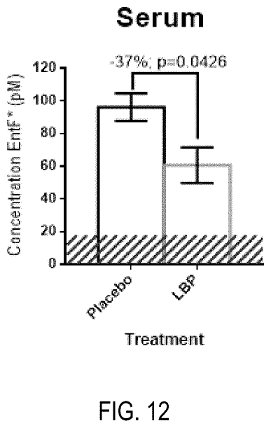

In the serum of the placebo group an average concentration of 96 pM (SEM=8 pM) EntF-metabolite (EntF*) was determined. However, the level of EntF* decreased significantly with 37%, to 61 pM (SEM=11 pM), when mice were treated with the test mixture. In FIG. 12, the serum concentrations of both groups is shown.

### REFERENCES

1. Kopetz, S. New therapies and insights into the changing landscape of colorectal cancer. Nature Reviews Gastroenterology & Hepatology 2019, 16: 79-80.
2. Feng, Q., et al. Gut microbiome development along the colorectal adenoma-carcinoma sequence. Nature Communications 2015, 6: 6528.
3. Louis, P., Hold, G. L., Flint, H. J. The gut Microbiota, bacterial metabolites and colorectal cancer. Nature Reviews Microbiology 2014, 12: 661-672.
4. Dinan, T. G. Brain-gut-microbiota axis—mood, metabolism and behaviour. Nature Reviews Gastroenterology & Hepatology 2017, 14: 69-70.
5. Tanoue T. et al. A defined commensal consortium elicits CD8 T cells and anti-cancer immunity. Nature 565, 600-605 (2019).
6. Janssens Y. et al. Disbiome database: linking the microbiome to disease. BMC Microbiology 18:50, 1-6 (2018).
7. Lu, Y., et al., Mucosal adherent bacterial dysbiosis in patients with colorectal adenomas. Scientific reports, 2016. 6: p. 26337.
8. Wang, T., et al. Structural segregation of gut microbiota between colorectal cancer patients and healthy volunteers. The ISME Journal 2012, 6: 320-329.
9. Kang, S., et al. Dysbiosis of fecal microbiota in Crohn's disease patients as revealed by a custom phylogenetic microarray. Inflammatory Bowel Diseases 2010, 16: 2034-2042.
10. Pascal, V., Pozuelo, M., Borruel, N., Casellas, F., Campos, D., et al. A microbial signature for Crohn's disease. Gut 2017, 66: 813-822.
11. Ni, J., et al. Gut microbiota and IBD: causation or correlation? Nature reviews Gastroenterology & Hepatology 2017, 14: 573-584.
12. Darkoh, C., et al. Toxin synthesis by Clostridium difficile is regulated through quorum signaling. mBio 2015, 6.
13. Wynendaele, E., et al. Quorumpeps database: chemical space, microbial origin and functionality of quorum sensing peptides. Nucleic Acids Research 2013, 41(Database issue): D655-659.
14. van Belkum, M. J., et al. Structure function relationship of inducer peptide pheromones involved in bacteriocin production in Carnobacterium maltaromaticum and Enterococcus faecium. Microbiology 2007, 153: 3660-3666.
15. Franz, C. M., et al. Characterization of the genetic locus responsible for production and immunity of carnobacteriocin A: the immunity gene confers cross-protection to enterocin B. Microbiology 2000, 146: 621-631.
16. Nilsen, T., I. F. Nes, and H. Holo. An exported inducer peptide regulates bacteriocin production in Enterococcus faecium CTC492. Journal of Bacteriology 1998, 180: 1848-1854.
17. Aymerich, T., et al. Biochemical and genetic characterization of enterocin A from Enterococcus faecium, a new antilisterial bacteriocin in the pediocin family of bacteriocins. Applied and Environmental Microbiology 1996, 62: 1676-1682.
18. Wynendaele, E., et al. Crosstalk between the microbiome and cancer cells by quorum sensing peptides. Peptides 2015, 64: 40-48.
19. De Spiegeleer, B., et al. The quorum sensing peptides PhrG, CSP and EDF promote angiogenesis and invasion of breast cancer cells in vitro. PloS one 2015, 10: e0119471.
20. Tommelein, J., et al. Radiotherapy-activated cancer-associated fibroblasts promote tumor progression through paracrine IGF1R activation. Cancer Research 2018, 79: 659-670.
21. Fumagalli, A., et al. A surgical orthotopic organoid transplantation approach in mice to visualize and study colorectal cancer progression. Nature protocols 2018, 13: 235-247.
22. Smith, M. D., & McCall, J. L. Systematic review of tumour number and outcome after radical treatment of colorectal liver metastases. Br J Surg 2009, 96: 1101-1113.
23. Xu, L. H., Cai, S. J., Cai, G. X. et al. Imaging diagnosis of colorectal liver metastases. World J Gastroenterol 2011, 17: 4654-4659.
24. Nguyen, K. T., Laurent, A., Dagher, I., Geller, D. A., Steel, J., Thomas, M. T., Marvin, M., Ravindra, K. V., Mejia, A., Lainas, P., Franco, D., Cherqui, D., Buell, J. F., & Gamblin, T. C. Minimally invasive liver resection for metastatic colorectal cancer: a multi-institutional, international report of safety, feasibility, and early outcomes. Ann Surg 2009, 250: 842-848.
25. Holch, J. W., Demmer, M., Lamersdorf, C., Michl, M., Schulz, C., von Einem, J. C., Modest, D. P., Heinemann, V. Pattern and dynamics of distant metastases in metastatic colorectal cancer. Visceral Medicine 2017, 33: 70-75.
26. Hubatsch, I., Ragnarsson, E. G. E., Artursson, P. Determination of drug permeability and prediction of drug absorption in Caco-2 monolayers. Nature Protocols 2007, 2: 2111-2119.

1-12. (canceled)

13. A method for reducing metastasis of colorectal cancer in a subject, the method comprising:
administering to the subject a microorganism that
reduces or blocks activity of a pro-metastatic quorum-sensing peptide or a metabolite thereof present in a gastrointestinal tract or blood of the subject; and/or that
reduces or blocks production of the pro-metastatic quorum-sensing peptide or the metabolite thereof in the gastrointestinal tract or blood of the subject.

14. The method of claim 13, wherein the pro-metastatic quorum-sensing peptide comprises EntF (SEQ ID NO:2) or the metabolite EntF* (SEQ ID NO:1) of EntF.

15. The method of claim 13, wherein the microorganism is selected from the group consisting of engineered microorganisms and bacterial strains, wherein:
the engineered microorganisms are unable to synthesize pro-metastatic quorum sensing peptides, and/or the engineered microorganisms reduce or inhibit functionality of the pro-metastatic quorum-sensing peptide or the metabolite; and
the bacterial strains are unable to synthesize the pro-metastatic quorum sensing peptide.

16. The method of claim 13, wherein the microorganism comprises engineered bacteria, wherein:
the engineered bacteria are unable to synthesize pro-metastatic quorum sensing peptides; and/or
the engineered bacteria reduce or inhibit functionality of the pro-metastatic quorum-sensing peptide or the metabolite.

17. The method of claim 13, wherein the microorganism comprises bacterial strains that are unable to synthesize the pro-metastatic quorum sensing peptide and are able to reduce or inhibit the production of the pro-metastatic quorum-sensing peptide by gut microbiota.

18. The method of claim 13, wherein:
the microorganism is a bacterial strain that does not produce EntF*; or
the microorganism is a bacterial strain that lacks EntF genes.

19. The method of claim 18, wherein the microorganism is a bacterial strain from order Lactobacillales.

20. The method of claim 19, wherein the bacterial strain is from genus Enterococcus or genus Lactococcus.

21. The method of claim 20, wherein the bacterial strain is E. faecium that is free of EntF and EntF* as determined by at least one of qPCR or UHPLC-MS/MS.

22. The method of claim 21, wherein the bacterial strain is selected from the group consisting of E. faecium LMG 15710, E. faecium NCIMB 10415 (SF68), E. faecium W54, E. faecium LMG S-28935, and E. faecium THT020101.

23. The method of claim 18, wherein:
the microorganism is a bacterial strain that does not produce EntF*; and
the bacterial strain reduces production of EntF* by other bacterial strains in the gastrointestinal tract of the subject by altering bacterial flora and/or by suppressing levels of bacterial strains that produce EntF/EntF*.

24. The method of claim 23, whereby the bacterial strain reduces or prevents a presence of bacterial strains producing EntF*.

25. The method of claim 13, wherein the subject is a human.

26. An in vitro method for detecting a quorum-sensing peptide or metabolite thereof in a sample from a subject, the method comprising:
performing C18 or hydrophilic interaction liquid chromatography solid-phase extraction on the sample to obtain a prepared sample;
performing liquid chromatography on the prepared sample under gradient conditions that minimize loss of an analyte; and
detecting the quorum-sensing peptide or metabolite thereof from the prepared sample by a mass spectrometry-based method after performing liquid chromatography.

27. The method of claim 26, further comprising:
choosing a start mobile phase, a gradient slope, or both, to establish the gradient conditions that minimize loss of the analyte.

28. The method of claim 27, wherein the gradient conditions comprise a start mobile phase of water:acetonitrile:dimethylsulfoxide containing from 0.05% to 1% by volume of an acid.

29. The method of claim 26, wherein the subject is a subject diagnosed with cancer.

30. The method of claim 26, wherein the subject is a subject diagnosed with colorectal cancer.


SEQUENCE LISTING (12 sequences)

SEQ ID NO: 1 - protein, 14 aa, Artificial Sequence
  EntF metabolite
SNLVECVFSLKKCN

SEQ ID NO: 2 - protein, 25 aa, Artificial Sequence
  EntF peptide
AGTKPQGKPASNLVECVFSLFKKCN

SEQ ID NO: 3 - protein, 48 aa, Artificial Sequence
  Enterocin induction factor
MEEKNRLNAKQCSDQELKKIKGGAGTKPQGKPASNLVECVFSLFKKCN

SEQ ID NO: 4 - protein, 15 aa, Artificial Sequence
  Antagonistic peptide
  VARIANT 1: X can be any amino acid other than S
  VARIANT 2: X can be any amino acid other than N
  VARIANT 10: X can be any amino acid other than L
XXLVECVFSXFKKCN

SEQ ID NO: 5 - protein, 15 aa, Artificial Sequence
  QSP76S1A
ANLVECVFSLFKKCN

SEQ ID NO: 6 - DNA, 22 bp, Artificial Sequence
  EntF metabolite Fw primer
acctcaaggaaaaccggcatca

SEQ ID NO: 7 - DNA, 22 bp, Artificial Sequence
  EntF metabolite Rev primer
tcaagagatcccctcctgattt

SEQ ID NO: 8 - DNA, 26 bp, Artificial Sequence
  EntF peptide Fw primer
tgctaaacaatgttcagatcaagagc

SEQ ID NO: 9 - DNA, 26 bp, Artificial Sequence
  EntF peptide Rev primer
cccacgaacattcttaatagataagc

SEQ ID NO: 10 - DNA, 144 bp, Artificial Sequence
  Enterocin induction factor nucleotide seq
atggaagaaaaaaatagactaaatgctaaacaatgttcagatcaagagcttaaaaaaattaaaggaggtgctggaacaaaacctcaaggaaaaccggcatcaaatttagtggaatgtgttttttcattatttaaaaagtgtaac

SEQ ID NO: 11 - DNA, 75 bp, Artificial Sequence
  EntF nucleotide seq
gctggaacaaaacctcaaggaaaaccggcatcaaatttagtggaatgtgttttttcattatttaaaaagtgtaac

SEQ ID NO: 12 - DNA, 45 bp, Artificial Sequence
  EntF metabolite nucleotide seq
tcaaatttagtggaatgtgttttttcattatttaaaaagtgtaac